(19) United States
(12) Patent Application Publication
(10) Pub. No.: US 2026/0098275 A1
(43) Pub. Date: Apr. 9, 2026

(54) UORF EDITING TO IMPROVE PLANT TRAITS

(71) Applicant: Inari Agriculture Technology, Inc., Cambridge, MA (US)

(72) Inventors: Ross Everett ALTMAN, Cambridge, MA (US); Malachy T. CAMPBELL, Cambridge, MA (US); Karl Anton Grothe KREMLING, Cambridge, MA (US); Ruijuan LI, Cambridge, MA (US)

(73) Assignee: Inari Agriculture Technology, Inc., Cambridge, MA (US)

(21) Appl. No.: 19/474,124

(22) PCT Filed: Apr. 10, 2024
(86) PCT No.: PCT/US2024/023821

§ 371 Date: Oct. 9, 2025

Related U.S. Application Data
(60) Provisional application No. 63/458,605, filed on Apr. 11, 2023.

(51) Int. Cl. C12N 15/82; C12N 9/22; C12N 15/11

(52) U.S. Cl. CPC C12N 15/8261; C12N 9/226; C12N 15/111; C12N 15/8201; C12N 2310/20

(57) ABSTRACT

Disclosed herein are methods of improving agronomic traits in crop plants through editing sequences within the 5′ untranslated region (UTR) of plant genomes.

### CROSS-REFERENCE TO RELATED APPLICATIONS

This application claims the benefit of U.S. Provisional Application No. 63/458,605, filed Apr. 11, 2023, hereby incorporated by reference in its entirety.

### BACKGROUND OF THE INVENTION

There is ongoing demand to improve important agronomic traits in crop plants. Producing superior crops or crops with novel traits is critical to addressing local and global challenges to supply in terms of food, employment, economic value, and fuel. Recent advances in genome editing technologies have provided opportunities for precise modification of the genome in many crop plants. For example, technologies based on genome editing proteins, such as zinc finger nucleases, TALENs, and CRISPR systems are advancing rapidly, and it is now possible to target genetic changes to specific DNA sequences in the genome.

Leader sequences precede the coding regions in the mRNA of eukaryotes and are referred to as the 5′ untranslated region (5′ UTR). Many important agronomic traits are partially regulated through cis-acting upstream open reading frames (uORFs), which are translation start/stop sites and associated sequences located in the 5′ untranslated region (UTR) of the coding sequences. Genome-wide bioinformatic analysis has indicated that over 50% of human mRNAs and about 35% of Arabidopsis thaliana mRNAs contain at least one putative uORF. The translation of uORFs generally inhibits the translation of the coding region, or the primary open reading frame (pORF), due to ribosomal stalling or introducing challenges to ribosomal initiation at the pORF translation start site. All uORFs contain at least one start codon and one stop codon. Many important agronomic traits are produced through the translation of pORFs, so one way to influence the level of pORF translation is to alter the sequence of uORFs. Recent studies have shown that editing uORFs can be employed to optimize the translation of genes in valuable plant phenotypes.

### SUMMARY OF THE INVENTION

The present disclosure focuses on altering protein level through manipulation of uORF sequences. Moreover, the present disclosure details the use of these changes in improving agronomic traits. Further disclosed are methods of using gene editing technology for enacting these changes in uORF sequences.

In one aspect, a method of improving an agronomic trait in a plant or part thereof is provided. In some embodiments, the method includes providing a gene editing system to the plant or part thereof, wherein the gene editing system edits a target site, wherein the target site includes nucleic acid encoding a translation start site for an upstream open reading frame (uORF) located 5′ to a translation start site for a primary open reading frame (pORF), wherein editing by the gene editing system results in a deletion, substitution, or insertion at the nucleic acid encoding the uORF translation start site.

Also provided is a method of increasing translation of a primary open reading frame (pORF) in a plant or part thereof, the method including providing a gene editing system to the plant or part thereof, wherein the gene editing system edits a target site, wherein the target site includes nucleic acid encoding a translation start site for an upstream open reading frame (uORF) located 5′ to a translation start site for the primary ORF, wherein editing by the gene editing system results in a deletion, substitution, or insertion at the nucleic acid encoding the uORF translation start site. In some embodiments, the editing by the gene editing system results in decreased translation of the uORF. In some embodiments, protein level of the protein encoded by the primary ORF is increased.

Also provided is a method of improving an agronomic trait in a plant, the method including providing a gene editing system to the plant or part thereof, wherein the gene editing system edits a target site, wherein the target site includes nucleic acid encoding a translation start site for an upstream open reading frame (uORF) located 5′ to a translation start site for a primary open reading frame (pORF), wherein editing by the gene editing system introduces a translation start site for an upstream open reading frame (uORF) in the target site. Also provided is a method of decreasing translation of a primary open reading frame (ORF) in a plant including providing a gene editing system, wherein the gene editing system edits a target site, wherein the target site includes nucleic acid encoding a translation start site for an upstream open reading frame (uORF) located 5′ to a translation start site for a primary open reading frame (pORF), wherein editing by the gene editing system introduces a translation start site for an upstream open reading frame (uORF) in the target site. In some embodiments, editing by the gene editing system results in increased translation of the uORF. In some embodiments, protein level of the protein encoded by the primary ORF is decreased.

In some embodiments, the gene editing system includes a precise base editing (PBE) system, a PRIME editing system, a CRISPR-Cas9 system, a CRISPR-Cpf1 system, homing endonucleases, a meganuclease, a zinc finger nuclease system, or a transcription activator-like effector nuclease (TALEN) system. In some embodiments, the gene editing system includes a Cas endonuclease and a guide RNA for the Cas endonuclease. In some embodiments, editing by the gene editing system results in translation of a peptide or polypeptide from the new translation start site.

In some embodiments, the primary ORF encodes a protein involved in drought tolerance, disease resistance, pest resistance, stress tolerance, yield, shape, odor, texture, metabolite production, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception. In some embodiments, the agronomic trait includes drought tolerance, disease resistance, pest resistance, stress tolerance, yield, shape, odor, texture, metabolite production, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception.

In some embodiments, the uORF translation start site is within 500 base pairs of the translation start site for the primary ORF. In some embodiments, the uORF translation start site includes a nucleotide sequence selected from the group consisting of AUG, ACG, CUG, UUG, AUA, and AUC. In some embodiments, the primary ORF translation start site includes a nucleotide sequence selected from the group consisting of AUG, ACG, CUG, UUG, AUA, and AUC. In some embodiments, the uORF translation start site is in-frame with the primary ORF translation start site. In some embodiments, the uORF translation start site is out-of-frame with the primary ORF translation start site.

In some embodiments, low or high levels of protein produced by the primary ORF are associated with an agronomically undesirable phenotype. In some embodiments, low or high levels of protein produced by the primary ORF are associated with an agronomically desirable phenotype.

In some embodiments, two or more, three or more, four or more, or five or more guide RNAs are provided to the plant as part of the gene editing system. In some embodiments, each guide RNA is complementary to a target sequence in a target site, wherein each target site includes a nucleic acid encoding a uORF translation start site located 5′ to a translation start site for a primary ORF. In some embodiments, each target site is located 5′ to the translation start site of the same primary ORF. In some embodiments, each target site is located 5′ to the translation start site of different primary ORFs. In some embodiments, two or more, three or more, four or more, or five or more uORFs are introduced into the plant or part thereof. In some embodiments, two or more, three or more, four or more, or five or more uORFs are removed.

In some embodiments, the gene editing system includes a Cas endonuclease is selected from the group consisting of Cas9, Cas12a (Cpf1), Cas12e (CasX), Cas12d (CasY), C2c1, C2c2, C2c3, Cas12h, Cas12i, Cas12j, Cas14, and an engineered Cas nuclease.

In some embodiments, a donor template nucleic acid is provided. In some embodiments, the donor template nucleic acid is inserted at the target site. In some embodiments, insertion of the donor template nucleic acid results in removal of a uORF translation start site. In some embodiments, insertion of the donor template nucleic acid results in insertion of a new translation start site.

In some embodiments, the Cas endonuclease is a Cas nickase. In some embodiments, the Cas nickase includes a mutation in one or more nuclease active sites. In some embodiments, the Cas nickase is associated with a reverse transcriptase. In some embodiments, the Cas nickase is fused to the reverse transcriptase.

In some embodiments, the guide RNA includes at its 3′ end a priming site and an edit to be incorporated into the genomic target. In some embodiments, the guide RNA includes a spacer sequence that is complementary to the target sequence and a protospacer adjacent motif (PAM) sequence. In some embodiments, the PAM sequence is located 1 to 30 nucleotides 5′ of the spacer sequence. In some embodiments, the PAM sequence includes the nucleotide sequence TT, TTT, TTAT, TTTN, TTGT, CTT, TTC, CC, NGG, or a T- or C-rich sequence, wherein the nucleotide N represents any nucleobase.

In some embodiments, the plant or part thereof is a crop plant. In some embodiments, the plant or part thereof is a monocot or a dicot. In some embodiments, the plant or part thereof is selected from the group consisting of maize, rice, sorghum, rye, barley, wheat, pearl millet, foxtail millet, flax, oats, sugarcane, turfgrass, switchgrass, soybean, canola, alfalfa, sunflower, cotton, tobacco, tomato, peanut, potato, cannabis, tomato, a forage crop, an industrial crop, a woody crop, a biomass crop, and Arabidopsis.

In some embodiments, the gene editing system is provided to the plant by transforming the plant with a vector including nucleic acid encoding a guide RNA and nucleic acid encoding an endonuclease. In some embodiments, the vector is a T-DNA.

In some embodiments, the nucleic acid encoding the guide RNA and the nucleic acid encoding the endonuclease are operably linked to a promoter. In some embodiments, the nucleic acid encoding the guide RNA and the nucleic acid encoding the endonuclease are operably linked to different promoters. In some embodiments, the promoter is inducible or constitutive. In some embodiments, the promoter is selected from the group consisting of CaMV35S, ubiquitin, Rsyn7, NOS, MAS, ALS, pEMU, AtU3, AtU6, OsU3, OsU6, Pol II, Pol III, a tissue-specific promoter, and a cell-specific type promoter.

In some embodiments, the plant or part thereof includes a leaf, a shoot, a meristem, a stem, or a root. In some embodiments, the gene editing system is provided to the plant by application of a composition including the gene editing system to the plant or part thereof. In some embodiments, the gene editing system is provided to the plant by spraying the plant with the composition including the gene editing system. In some embodiments, the composition including the gene editing system includes a surfactant. In some embodiments, the composition including the gene editing system includes glass beads coating the guide RNA. In some embodiments, application of the gene editing system includes rubbing a composition including the gene editing system onto the leaves, shoot, stem, and/or meristem. In some embodiments, application of the gene editing system includes injecting a composition including the gene editing system into the stem. In some embodiments, application of the gene editing system includes leaf infiltration of a composition including the gene editing system into the leaf. In some embodiments, the leaf infiltration includes forced infiltration using a needle-less syringe or vacuum pump. In some embodiments, the composition including the gene editing system includes a nuclease inhibitor. In some embodiments, the nuclease inhibitor includes an RNase inhibitor. In some embodiments, application includes biolistic transformation of nucleic acid encoding the gene editing system into a leaf, shoot, shoot, stem, and/or meristem of the plant or part thereof. In some embodiments, the biolistic transformation includes transformation of circular DNA encoding the gene editing system.

In some embodiments, each of a plurality of plants is provided with a gene editing system, wherein editing by the gene editing system creates an allelic series.

In some embodiments, improving an agronomic trait in a plant further includes retrieving a progeny of the plant, wherein the progeny has an edited target sequence. In some embodiments, an edited plant is produced. In some embodiments, progeny is produced by the edited plant.

### DETAILED DESCRIPTION OF THE INVENTION

One aspect of the present disclosure includes methods of increasing protein expression or improving agronomic traits through the inhibition of an upstream open reading frame (uORF). An uORF is a cis-regulatory genetic element found widely across living organisms, defined by its position upstream to a primary open reading frame (pORF); this arrangement positions uORFs in 5′ untranslated regions of eukaryotic mRNAs and often results in their translation affecting the translation efficiency of downstream pORFs. Like pORFs, uORFs have translation start sites to which translational machinery binds, often comprising nucleotide bases AUG. Accordingly, in some embodiments, the uORF translation start site comprises the nucleotide sequence AUG, ACG, CUG, UUG, AUA, or AUC. In some embodiments, the uORF translation start site comprises the nucleotide sequence AUG. In some embodiments, the uORF translation start site encodes the amino acid methionine. In some embodiments, the uORF translation start site encodes the amino acid threonine. In some embodiments, the uORF translation start site encodes the amino acid leucine. In some embodiments, the uORF translation start site encodes the amino acid isoleucine. In some embodiments, the primary ORF translation start site includes a nucleotide sequence selected from the group consisting of AUG, ACG, CUG, UUG, AUA, and AUC.

TABLE 1
Codon table displaying the translated amino acids resulting
from each possible start or stop codon. When relevant, both
the RNA (Uracil) and DNA (Thymine) version is shown.

| Type of codon | Nucleotide code | Translated Codon |
|---|---|---|
| Start codons | AUG/ATG | Methionine |
| | ACG | Threonine |
| | CUG/CTG | Leucine |
| | UUG/TTG | Leucine |
| | AUA/ATA | Isoleucine |
| | AUC/ATC | Isoleucine |
| Stop codons | UAA/TAA | Stop (Ochre) |
| | UGA/TGA | Stop (Opal) |
| | UAG/TAG | Stop (Amber) |

The size and location of the uORF can affect its activity. An uORF is generally considered to be short and only requires a start codon and stop codon. Beyond this minimum length, uORF lengths can vary. In yeast, for example, the majority of uORFs over 3 codons in length were shorter than 20 codons in length, but the mean was 16 codons (Lawless et al. (2009) BMC Genomics 10:7). In cereal crops (rice, wheat, barley, maize, and sorghum), 51% of all uORFs have been found to be under 20 codons in length (Tran et al. (2008) BMC Genomics 9, 361), but median uORF length is 22 nucleotides in the Brassica plant Arabidopsis thaliana and 477 nucleotides in the bryophyte Physcomitrella patens (Zhang et al. (2021) Nat Commun. 12(1):1076. Erratum in: (2021) Nat Commun. 12(1): 2101). While shorter uORFs are more common, longer ones also occur.

Accordingly, in some embodiments, the uORF is between 6 and 700 base pairs in length, for example about 6 to 15 base pairs, 6 to 45 base pairs, 6 to 60 base pairs, 6 to 75 base pairs, 6 to 120 base pairs, 6 to 150 base pairs, 6 to 200 base pairs, 6 to 300 base pairs, 6 to 400 base pairs, 6 to 500 base pairs, 6 to 600 base pairs, and up to 700 base pairs. In some embodiments, the uORF is between 700 and 1000 base pairs. In some embodiments, the uORF extends 0 to 100 base pairs into the pORF, for example 0 to 10 base pairs, 0 to 25 base pairs, 0 to 50 base pairs, 0 to 75 base pairs, and up to about 100 base pairs.

Accordingly, in some embodiments, the uORF encodes between 1 and 230 amino acids, for example about 1 to 6 amino acids, 1 to 15 amino acids, 1 to 20 amino acids, 1 to 30 amino acids, 1 to 40 amino acids, 1 to 50 amino acids, 1 to 60 amino acids, 1 to 75 amino acids, 1 to 80 amino acids, 1 to 90 amino acids, 1 to 100 amino acids, 1 to 120 amino acids, 1 to 135 amino acids, 1 to 150 amino acids, 1 to 165 amino acids, 1 to 175 amino acids, 1 to 190 amino acids, 1 to 200 amino acids, 1 to 215 amino acids, and up to about 1 to 230 amino acids.

In some embodiments, the spacing between the uORF and pORF can vary. In some embodiments, the uORF is located 5′ to the pORF. In some embodiments, the uORF translation start site is located 5′ to the pORF translation start site. In some embodiments, the uORF and pORF overlap. While there may be intervening nucleotides between the uORF in the 5′ UTR and the translational start site of the pORF, some uORFs instead have a translation stop site overlapping with nucleotides within the pORF. The spacing between the uORF translation stop site and the pORF translation start site is referred to as the “intercistronic distance” or “intercistronic spacer.” In certain circumstances, regulation of the pORF by the uORF has been demonstrated to be more efficient when the intercistronic distance is shorter rather than larger (Kochetov et al. (2008) FEBS Letters, 582(9): 1293-1297; Luukkonen et al. (1995) Journal of Virology 69(7): 4086-4094). Accordingly, in some embodiments, the uORF translation start site is within 500 base pairs of the translation start site for the primary ORF, for example, 1 to 50 base pairs, 1 to 100 base pairs, 1 to 150 base pairs, 1 to 200 base pairs, 1 to 250 base pairs, 1 to 300 base pairs, 1 to 350 base pairs, 1 to 400 base pairs, 1 to 450 base pairs, or 1 to 500 base pairs. In some embodiments, the pORF translation start site is located within the uORF. In some embodiments, the uORF is directly adjacent to the translation start site. In some embodiments, the distance between the translation start site of the uORF and the translation start site of the pORF is 1 to about 500 base pairs, for example, 1 to 50 base pairs, 1 to 100 base pairs, 1 to 150 base pairs, 1 to 200 base pairs, 1 to 250 base pairs, 1 to 300 base pairs, 1 to 350 base pairs, 1 to 400 base pairs, 1 to 450 base pairs, or 1 to about 500 base pairs. In some embodiments, the distance between the translation stop site of the uORF and the translation start site of the pORF is about 1 to 500 base pairs, for example, 1 to 50 base pairs, 1 to 100 base pairs, 1 to 150 base pairs, 1 to 200 base pairs, 1 to 250 base pairs, 1 to 300 base pairs, 1 to 350 base pairs, 1 to 400 base pairs, 1 to 450 base pairs, or about 1 to 500 base pairs. In some embodiments, the spacing of the uORF relative to the pORF is in-frame. In some embodiments, the spacing of the uORF relative to the pORF is out-of-frame. Accordingly, in some embodiments, the uORF translation start site is in-frame with the primary ORF translation start site. In other embodiments, the uORF translation start site is out-of-frame with the primary ORF translation start site. If the uORF is in-frame with the pORF, both reading frames may share a stop codon. In some embodiments, the uORF stop codon is selected from the group consisting of UAA, UGA, and UAG.

In some embodiments, uORFs inhibit the translation of downstream pORFs. This inhibitory activity in plants is generally understood to be the product of ribosomal stalling or prompting the disassociation of ribosomes from the eukaryotic mRNA strand at the uORF stop site, with attenuated potential for the ribosomes to reinitiate at the pORF. Because successful translation of the uORF generally decreases the successful translation of the pORF under either model, the altering of the uORF sequence may decrease the rate of uORF translation and thereby increase the rate of pORF translation. Accordingly, in some embodiments, inducing change in the uORF sequence decreases the rate of uORF translation and increases the rate of pORF translation, thereby increasing the level of protein produced by the pORF. In some embodiments, editing the translation start codon of the uORF results in a decrease in uORF protein level of about 30% to 100%, for example, between 30% and 40%, 30% and 50%, 30% and 60%, 30% and 70%, 30% and 75%, 30% and 80%, 30% and 85%, 30% and 90%, 30% and 95%, and up to about 100%. In some embodiments, editing the translation start codon of the pORF results in an increase in the pORF protein level by about 30% to 500%, for example, between 30% and 40%, 30% and 50%, 30% and 60%, 30% and 70%, 30% and 75%, 30% and 80%, 30% and 85%, 30% and 90%, 30% and 95%, 30% and 100%, and up to about 125%, up to about 150%, up to about 175%, up to about 200%, up to about 225%, up to about 250%, up to about 275%, up to about 300%, up to about 325%, up to about 350%, up to about 375%, up to about 400%, up to about 425%, up to about 450%, up to about 475%, or up to about 500%.

Further explanation of uORFs and their relation to pORFs can be found in Campbell et al. (2019) Nature Scientific Reports, 9, 14757; Zhang et al. (2018) Nat. Biotech. 36(9): 894-898; Ferreira et al. (2013) PNAS 110(28): 11284-9; and Si et al. (2020) Nat. Protocols, 15: 338-363; which are herein incorporated by reference.

Gene Editing to Alter uORFs

In some embodiments of the present disclosure, the uORF sequence is changed to improve agronomic traits through gene editing techniques. In some embodiments, these gene editing techniques are used to modify one or more base pairs in the uORF in order to disable it, so it is no longer translatable. In some embodiments, the gene editing induces substitution of at least one base in an uORF start codon that prevents uORF translation. In some embodiments, the gene editing results in a deletion at the nucleic acid encoding the uORF start site, such that a uORF start codon is removed. In some embodiments, the gene editing results in an insertion such that a uORF start codon is removed. In some embodiments, the gene editing induces substitution of at least one base in an uORF that produces a premature stop codon, truncating uORF translation. In some embodiments, the uORF is edited to decrease its translation rate. In some embodiments, the uORF is edited to improve agronomic traits by decreasing translation rates of the uORF. In some embodiments, the uORF is modified to improve agronomic traits by decreasing the inhibition of the associated pORF(s). In some embodiments, the uORF is modified to improve agronomic traits by increasing the translation of the associated pORF(s). In some embodiments, the uORF is modified to improve agronomic traits by increasing the protein levels of the associated pORF(s).

The genetic editing of plants to improve agronomic traits may be accomplished through any method known to one skilled in the art, including methods such as homing endonucleases, meganucleases, zinc finger nucleases, and transcription activator-like effector nucleases (TALENs), which require de novo protein engineering for every new target locus. The highly specific, RNA-directed DNA nuclease, guide RNA/Cas9 endonuclease system described herein, is more easily customizable and therefore more useful when modification of many different target sequences is the goal. As known in the art, gene editing can enact dramatic genetic change in many fewer plant generations than traditional breeding methods. In some embodiments of the present disclosure, the editing technique is a CRISPR/Cas system.

Meganucleases are sequence-specific endonucleases with large (>14 bp) cleavage sites that can deliver DNA double-strand breaks (DSBs) at specific loci in living cells (Thierry and Dujon (1992) Nucleic Acids Res., 20: 5625-5631). Meganucleases have been used to stimulate homologous recombination in the vicinity of their target sequences in cultured cells and plants (Rouet (1994) Mol. Cell. Biol. 14: 8096-106; Choulika et al. (1995) Mol. Cell. Biol. 15: 1968-73; Donoho et al. (1998) Mol. Cell. Biol. 18: 4070-8; Elliott (1998) Mol. Cell. Biol. 18: 93-101; Sargent et al. (1997) Mol. Cell. Biol. 17: 267-77; Puchta et al. (1996) Proc. Natl. Acad. Sci. USA, 93: 5055-60; Chiurazzi et al. (1996) Plant Cell, 8: 2057-2066), making meganuclease-induced recombination an efficient and robust method for genome engineering. In nature, meganucleases are essentially represented by homing endonucleases (HEs), a family of endonucleases encoded by mobile genetic elements, whose function is to initiate DNA double-strand break (DSB)-induced recombination events in a process referred to as homing (Chevalier and Stoddard (2001) Nucleic Acids Res. 29: 3757-74; Kostriken et al. (1983) Cell, 35: 167-74; Jacquier and Dujon (1985) Cell, 41: 383-94).

“CRISPR (Clustered Regularly Interspaced Short Palindromic Repeats)/Cas (CRISPR-associated) systems,” or CRISPR systems, are adaptive defense systems originally discovered in bacteria and archaea. CRISPR systems use RNA-guided nucleases termed CRISPR-associated or “Cas” endonucleases (e.g., Cas9 or Cas12a (“Cpf1”)) to cleave foreign DNA. In a typical CRISPR/Cas system, a Cas endonuclease is directed to a target nucleotide sequence (e.g., a site in the genome that is to be sequence-edited) by sequence-specific, non-coding “guide RNAs” that target single- or double-stranded DNA sequences. In some embodiments, the target is within the 5′ UTR of a eukaryote. In some embodiments, the target is within the 5′ UTR of a plant. In some embodiments, the target is within the 5′ UTR of a crop plant.

It is envisioned by the inventors that embodiments of the present disclosure can span multiple types of CRISPR/Cas systems. In some embodiments, the Cas associated with the CRISPR/Cas system is a Cas nickase. Two classes (1 and 2) of CRISPR systems have been identified across a wide range of bacterial hosts. The well characterized class 2 CRISPR systems use a single Cas endonuclease (rather than multiple Cas proteins). One class 2 CRISPR system includes a type II Cas endonuclease such as Cas9, a CRISPR RNA (“crRNA”), and a trans-activating crRNA (“tracrRNA”). A “trans-activating crRNA” or “tracrRNA” is a trans-encoded small RNA that is partially homologous to repeats within a CRISPR array. At least in the case of Cas9 type CRISPR systems, both a tracrRNA and a crRNA are required for the CRISPR array to be processed and for the nuclease to cleave the target DNA sequence. In contrast, Cas12a type CRISPR systems have been reported to function without a tracrRNA, with the Cas12a CRISPR arrays processed into mature crRNAs without the requirement of a tracrRNA; see Zetsche et al. (2015) Cell, 163:759-771 and U.S. Pat. No. 9,790,490. The Cas9 crRNA contains a “spacer sequence”, typically an RNA sequence of about 20 nucleotides (this may be 20 to about 30 contiguous nucleotides in length, such as 20 to 21, 20 to 22, 20 to 23, 20 to 24, 20 to 25, or up to about 30 contiguous nucleotides in length) that corresponds to (e.g., is identical or nearly identical to, or alternatively is complementary or nearly complementary to) a target DNA sequence of about equivalent length. The Cas9 crRNA also contains a region that binds to the Cas9 tracrRNA to form a partially double-stranded structure which is cleaved by RNase III, resulting in a crRNA:tracrRNA hybrid or duplex. The crRNA:tracrRNA hybrid then directs the Cas9 endonuclease to recognize and cleave the target DNA sequence; in some examples, a tracrRNA and crRNA (e.g., a crRNA including a spacer sequence) can be included in a chimeric nucleic acid referred to as a “single guide RNA” (sgRNA).

The Cas12a (“Cpf1”) CRISPR system includes the type V endonuclease Cas12a (also known as “Cpf1”). Cas12a nucleases are characterized as having only a RuvC nuclease domain, in contrast to Cas9 nucleases which have both RuvC and HNH nuclease domains. Cas12a nucleases are generally smaller proteins than Cas9 nucleases and can function with a smaller guide RNA (e.g., a crRNA having at least one spacer flanked by direct repeats), which are practical advantages in that the nuclease and guide RNAs are more economical to produce and potentially more easily delivered to a cell. Examples of Cas12a nucleases include AsCas12a or “AsCpf1” (from Acidaminococcus sp.) and LbCas12a or “LbCpf1” (from Lachnospiraceae bacteria). In contrast to Cas9 type CRISPR systems, Cas12a-associated (“Cpf1”-associated) CRISPR arrays have been reported to be processed into mature crRNAs without the requirement of a tracrRNA, i.e., the naturally occurring Cas12a (Cpf1) CRISPR system was reported to require only the Cas12a (Cpf1) nuclease and a Cas12a crRNA to cleave the target DNA sequence; see Zetsche et al. (2015) Cell, 163:759-771; U.S. Pat. No. 9,790,490. Accordingly, in some embodiments, the Cas endonuclease is selected from the group consisting of Cas9, Cas12a (Cpf1), Cas12e (CasX), Cas12d (CasY), C2c1, C2c2, C2c3, Cas12h, Cas12i, Cas12j, Cas14, and an engineered Cas nuclease. In some embodiments, the Cas nickase includes a mutation in one or more nuclease active sites. In some embodiments, the Cas nickase is associated with a reverse transcriptase. In some embodiments, the Cas nickase is fused to the reverse transcriptase. As used herein, the term “reverse transcriptase” (i.e., RNA-directed DNA polymerases) refers to a group of enzymes having reverse transcriptase activity (i.e., that catalyze synthesis of DNA from an RNA template). In general, such enzymes include, but are not limited to, retroviral reverse transcriptase, retrotransposon reverse transcriptase, and bacterial reverse transcriptases such as group II intron-derived reverse transcriptase, and mutants, variants, or derivatives thereof.

For the purposes of gene editing, CRISPR arrays can be designed to contain one or multiple guide RNAs designed to target a DNA sequence for editing, where the guide RNA includes at least one spacer sequence that corresponds to a specific locus of about equivalent length in the target DNA; see, for example, Cong et al. (2013) Science, 339:819-823; Ran et al. (2013) Nature Protocols, 8:2281-2308. Depending on the CRISPR/Cas system used, there are multiple configurations available for guide RNA. These various constructions of the guide RNA, as well as how to design them, are well known to one skilled in the art. As used herein, the terms “guide RNA” or “gRNA” refer to a nucleic acid that comprises or includes a nucleotide sequence (sometimes referred to as a “spacer sequence”) that corresponds to (e.g., is identical or nearly identical to, or alternatively is complementary or nearly complementary to) a target DNA sequence (e.g., a contiguous nucleotide sequence that is to be modified) in a genome; the guide RNA functions in part to direct the CRISPR nuclease to a specific location on the genome. In embodiments, a gRNA is a CRISPR RNA (“crRNA”). For nucleases (such as a Cas9 nuclease) that require a combination of a trans-activating crRNA (“tracrRNA”) and a crRNA for the nuclease to cleave the target nucleotide sequence, the gRNA can be a tracrRNA:crRNA hybrid or duplex, or can be provided as a single guide RNA (sgRNA). At least 16 or 17 nucleotides of gRNA sequence corresponding to a target DNA sequence are required by Cas9 for DNA cleavage to occur; for Cas12a (Cpf1) at least 16 nucleotides of gRNA sequence corresponding to a target DNA sequence are needed to achieve detectable DNA cleavage and at least 18 nucleotides of gRNA sequence corresponding to a target DNA sequence were reported necessary for efficient DNA cleavage in vitro; see Zetsche et al. (2015) Cell, 163:759-771. Cas12a (Cpf1) endonuclease and corresponding guide RNAs are disclosed in U.S. Pat. No. 9,790,490, which is incorporated herein by reference in its entirety.

Accordingly, in some embodiments, a gene editing system binds to a target sequence within a target site that comprises a part of the uORF. In some embodiments, guide RNA binds to a target sequence within a target site that comprises a part of the uORF. In some embodiments, the gene editing system binds to a target sequence within a target site that comprises the start site of the uORF. In some embodiments, guide RNA binds to a target sequence within a target site that comprises the start site of the uORF. In some embodiments, the guide RNA and/or the endonuclease are provided to the plant by transforming the plant with a vector including nucleic acid encoding the guide RNA and nucleic acid encoding the endonuclease. In some embodiments, the gene editing system is provided to the plant by transforming the plant with a vector including nucleic acid encoding the gene editing system. In some embodiments, the guide RNA and/or the endonuclease are provided to the plant by transforming the plant with a vector including nucleic acid encoding the guide RNA and nucleic acid encoding the endonuclease. In some embodiments, the composition including the gene editing system includes a nuclease inhibitor. In some embodiments, the composition including the guide RNA includes a nuclease inhibitor. In some embodiments, the nuclease inhibitor includes an RNase inhibitor. In some embodiments, the nuclease inhibitor is a non-cleavable oligonucleotide, an aptamer, a DNP-Poly(A), a competitive inhibitor comprising a ribonucleoside, a deoxyribonucleoside, or a dideoxyribonucleoside. In some embodiments, the RNAse inhibitor is an RNase inhibitor protein (RIP), a protease, a tyrosine-glutamate copolymer, actin, or RraA. In some embodiments, the gene editing system includes a priming site and an edit to be incorporated into the genomic target. In some embodiments, the guide RNA of the gene editing system includes at its 3′ end a priming site and an edit to be incorporated into the genomic target. As used herein, the term “priming site” refers to a sequence complementary to the 3′ end of the target DNA cut site.

Across CRISPR/Cas systems, a Cas endonuclease associated with guide RNA(s) cuts at a priming site depending on the presence of a nearby Cas-recognizable sequence, called the “protospacer adjacent motif” (the PAM). The PAM sequences a Cas endonuclease can recognize depend on the specific Cas endonuclease used. Accordingly, in some embodiments, the guide RNA includes a spacer sequence that is complementary to the target sequence and a PAM sequence. In some embodiments, the PAM sequence is located 1 to 30 nucleotides 5′ of the spacer sequence, such as within 1 to 5 nucleotides, 1 to 10 nucleotides, 1 to 15 nucleotides, 1 to 20 nucleotides, 1 to 25 nucleotides, or 1 to 30 nucleotides. In some embodiments, the PAM sequence includes the nucleotide sequence TT, TTT, TTAT, TTTN, TTGT, CTT, TTC, CC, NGG, or a T- or C-rich sequence, wherein the nucleotide N represents any nucleobase. In some embodiments, the PAM sequence includes the nucleotide sequence TTTN.

One aspect of the present disclosure includes a method of improving an agronomic trait in a plant or part thereof, providing a Cas endonuclease and a guide RNA for the Cas endonuclease to the plant or part thereof, wherein the guide RNA is complementary to a target sequence in a target site, wherein the target site includes nucleic acid encoding a translation start site for an upstream open reading frame (uORF) located 5′ to a translation start site for a primary open reading frame (ORF), wherein editing by the Cas endonuclease and the guide RNA results in a deletion, substitution, or insertion at the nucleic acid encoding the uORF translation start site.

Experimental validation of CRISPR/Cas application can be examined on multiple levels. Validating the successful delivery of the CRISPR system into experimental cells, plants, or plant parts often involves testing antibiotic resistance or performing a detection assay. Detection can be achieved by amplification and/or hybridization-based detection methods using a method (e.g., selective amplification primers) and/or probe (e.g., capable of selective hybridization or generation of a specific primer extension product) which specifically recognizes the target DNA molecule (e.g., transgenic locus excision site) but does not recognize DNA from an unmodified transgenic locus. In certain embodiments, the hybridization probes can comprise detectable labels (e.g., fluorescent, radioactive, epitope, and chemiluminescent labels). In certain embodiments, a single nucleotide polymorphism detection assay can be adapted for detection of the target DNA molecule (e.g., transgenic locus excision site). A vector delivered to a plant or plant part thereof may include an antibiotic resistance gene, which then allows antibiotic treatment of CRISPR-treated plants or plant parts to identify surviving individuals as those successfully contacted by the CRISPR/Cas system. Other examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification. Confirming successful gene targeting can be validated through sequencing transformed plants or plant parts.

### Increasing Protein/Translation Levels in Plants

The manipulation of an uORF sequence can affect its translation level. A CRISPR/Cas system, for example, can produce blunt or staggered breaks, which are then repaired by processes that may introduce errors to the sequence, such as non-homologous end joining (NHEJ), a DSB repair process that does not rely on homology for re-ligating the free ends produced by the break. For multiple forms of gene editing, repeated rounds of editing are known in the art and can lead to a majority of cells possessing an edited form of the specific uORF targeted.

In some embodiments, an uORF is edited to result in insertion of at least one nucleotide, a deletion of at least one nucleotide, a substitution of at least one nucleotide (such as creation of a point mutation in the locus), and this substitution may lessen the frequency with which the uORF is recognized by the native translational machinery of the plant cell. This may result in either partial or full inhibition of the uORF's translation, therein allowing increased translation of the pORF. The pORF's increased translation results in higher levels of resultant pORF proteins. In some embodiments, editing by the gene editing system results in decreased translation of the uORF. In some embodiments, a gene editing system is used to induce substitution in at least one base of an uORF start codon that prevents uORF translation. In some embodiments, a CRISPR/Cas system is used to induce substitution in at least one base of an uORF start codon that prevents uORF translation. In some embodiments, editing by the gene editing system results in decreased translation of the uORF. In some embodiments, editing by the endonuclease and the guide RNA results in decreased translation of the uORF. In some embodiments, protein level of the protein encoded by the primary ORF is increased. In some embodiments, the uORF protein level is decreased by about 30% to 100%, for example, between 30% and 40%, 30% and 50%, 30% and 60%, 30% and 70%, 30% and 75%, 30% and 80%, 30% and 85%, 30% and 90%, 30% and 95%, and up to about 100%. In some embodiments, the pORF protein level is increased by about 30% to 500%, for example, between 30% and 40%, 30% and 50%, 30% and 60%, 30% and 70%, 30% and 75%, 30% and 80%, 30% and 85%, 30% and 90%, 30% and 95%, 30% and 100%, and up to about 125%, up to about 150%, up to about 175%, up to about 200%, up to about 225%, up to about 250%, up to about 275%, up to about 300%, up to about 325%, up to about 350%, up to about 375%, up to about 400%, up to about 425%, up to about 450%, up to about 475%, and up to about 500%. Target protein levels are measured through methods known to one skilled in the art, such as Western blotting, immunoprecipitation assays, enzyme-linked immunosorbent assays (ELISA), and other tests.

One aspect of the present disclosure includes a method of improving an agronomic trait in a plant or part thereof including providing a gene editing system to the plant or part thereof, wherein the gene editing system edits a target site, wherein the target site comprises nucleic acid encoding a translation start site for an upstream open reading frame (uORF) located 5′ to a translation start site for a primary open reading frame (pORF), wherein editing by the gene editing system results in a deletion, substitution, or insertion at the nucleic acid encoding the uORF translation start site. Another aspect of the present disclosure includes a method of improving an agronomic trait in a plant including providing an endonuclease and a guide RNA for the endonuclease to the plant or part thereof, wherein the guide RNA is complementary to a target sequence in a target site, wherein the target site is located 5′ to a translation start site for a primary open reading frame (ORF), wherein editing by the Cas endonuclease and the guide RNA results in a deletion, substitution, or insertion at the nucleic acid encoding the uORF translation start site. One aspect of the present disclosure includes a method of decreasing translation of an uORF in a plant or part thereof comprising providing a gene editing system to the plant or part thereof, wherein the gene editing system edits a target site, wherein the target site comprises nucleic acid encoding a translation start site for an upstream open reading frame (uORF) located 5′ to a translation start site for the primary ORF, wherein editing by the gene editing system results in a deletion, substitution, or insertion at the nucleic acid encoding the uORF translation start site. Another aspect of the present disclosure includes a method of decreasing translation of an uORF in a plant or part thereof including providing a Cas endonuclease and a guide RNA for the Cas endonuclease to the plant or part thereof, wherein the guide RNA is complementary to a target sequence in a target site, wherein the target site includes nucleic acid encoding a translation start site for an upstream open reading frame (uORF) located 5′ to a translation start site for the primary ORF, wherein editing by the Cas endonuclease and the guide RNA results in a deletion, substitution, or insertion at the nucleic acid encoding the uORF translation start site. One aspect of the present disclosure includes a method of increasing translation of a primary open reading frame (pORF) in a plant or part thereof comprising providing a gene editing system to the plant or part thereof, wherein the gene editing system edits a target site, wherein the target site comprises nucleic acid encoding a translation start site for an upstream open reading frame (uORF) located 5′ to a translation start site for the primary ORF, wherein editing by the gene editing system results in a deletion, substitution, or insertion at the nucleic acid encoding the uORF translation start site. Another aspect of the present disclosure includes a method of increasing translation of a primary open reading frame (ORF) in a plant or part thereof including providing a Cas endonuclease and a guide RNA for the Cas endonuclease to the plant or part thereof, wherein the guide RNA is complementary to a target sequence in a target site, wherein the target site includes nucleic acid encoding a translation start site for an upstream open reading frame (uORF) located 5′ to a translation start site for the primary ORF, wherein editing by the Cas endonuclease and the guide RNA results in a deletion, substitution, or insertion at the nucleic acid encoding the uORF translation start site.

### Producing Allelic Series

One potential utility of altering protein levels through uORF manipulation is the generation of an allelic series. As used herein, an “allelic series” refers to a range of phenotypes observable as a consequence of variable mutations across multiple alleles for at least one given gene. The ability to analyze multiple alleles for a gene is highly useful for determining the gene's function, especially for genes that are lethal in a homozygous state (Campbell et al. (2019) Nature Scientific Reports, 9, 14757) or that produce complex traits. Furthermore, a pattern emerging from shared alleles can assist in determining the evolutionary order of mutations (across a developmental timescale).

Gene editing technology provides new opportunity for producing allelic series, allowing the fine scale targeting of a gene of interest, either with one gene editing system or in a multiplex system. In one example, cleavages in a gene of interest caused by a CRISPR-Cas9 editing system may be imprecisely repaired in different ways across a genome, producing a number of different mutations. Accordingly, in some embodiments, a plurality of plants is provided with a gene editing system, wherein editing by the gene editing system creates an allelic series.

### Precise Base Editing

Targeted modification of sequences may also be accomplished through the use of precise base editing (PBE). The precise base editor system is a system that has recently been developed based on CRISPR-Cas9, which enables accurate single-base editing of a genome using a nuclease-inactivated fusion protein of Cas9 protein and cytidine deaminase. Nuclease-inactivated Cas9 (due to mutations in the HNH subdomain and/or RuvC subdomain of the DNA cleavage domain) retains gRNA-directed DNA-binding ability, and the cytidine deaminase can catalyze deamination of cytidine (C) on DNA to form uracil (U). The nuclease-inactivated Cas9 is fused with a cytidine deaminase. Under the guidance of the guide RNA, the fusion protein can target the target sequence in the plant genome. Due to the absence of the Cas9 nuclease activity, the DNA double strand is not cleaved. The deaminase domain in the fusion protein converts the cytidine of the single-stranded DNA produced in the formation of the Cas9-gRNA-DNA complex to U, and the substitution of C to T is achieved by base mismatch repair. The precise base editor system suitable for use in the present invention includes, but is not limited to, the system described in Zong et al. (2017) Nat. Biotechnol. 35(5): 438-440.

Accordingly, in some embodiments, a precise base editing system is used to target an uORF located 5′ to a translation start site for the primary ORF, and the use of this system results in a deletion, substitution, or insertion at the nucleic acid encoding the uORF translation start site. This modification of the uORF translation start site results in the decreased translation of the uORF, which thereby increases the translation of the pORF, and improves an agronomic trait of interest encoded by the pORF.

### PRIME Editing

The insertion of one or more sequences to form a translation start site may be accomplished through the use of PRIME editing (Anzalone et al. (2019) Nature, 576(7785): 149-157). Prime editing makes precise DNA sequence modifications rather than random insertions, deletions, and substitutions (Indels), thus increasing the probability of obtaining the desired effect. Prime editing may be used to introduce any single base pair substitution as well as small deletion or insertions. Deletions of up to 80 base pairs have been produced using prime editing with a single prime editing Cas guide RNA (pegRNA) in human cells, and insertions of up to 40 base pairs (Anzalone et al. (2019) Nature, 576: 149-157). Dual pegRNA systems are also known in the art (Choi et al. (2021) Nat. Biotechnol. 40(2): 218-226; Lin et al. (2021) Nat. Biotechnol. 39(8): 923-927) and can be used to generate precise large deletions, or to improve editing efficiency for small insertions, deletions, or substitutions. Additionally, dual pegRNA systems where the extension of the pegRNAs are not complementary to the endogenous locus, but are complementary to one another, can be used to replace endogenous sequence and/or mediate larger insertions (Anzalone et al. (2022) Nat. Biotechnol. 40(5): 731-740).

Prime editing can also be accomplished with Cas nucleases in place of Cas nickases (Adikusuma et al. (2021) Nucleic Acids Res. 49(18): 10785-10795). In some embodiments, prime editing uses (i) a Cas nuclease, in some embodiments a Cas9 nuclease, in other embodiments a Cas12 nuclease, fused to a reverse transcriptase (Cas-RT), in some embodiments a M-MLV reverse transcriptase, and (ii) a pegRNA that both specifies the genome target site and has an extension that encodes the target edit within a template for the reverse transcriptase. In some embodiments, the binding of the pegRNA directs the Cas nuclease to create a double-stranded break in the DNA at the target site. The extension of the pegRNA binds to the cut DNA that has an exposed 3′-hydroxyl group, priming the reverse transcriptase to produce a DNA strand that is complementary to the extension of the pegRNA. This DNA strand will include the complement to any desired edits present in the provided pegRNA extension. Mismatch repair by the cell will then resolve the mismatch between the unedited parent strand and the edited product of the reverse transcriptase, thus introducing the desired edits into the genome. Prime editing systems may also include elements to inhibit mismatch repair, or to nick the unedited parent strand to increase editing efficiency.

Accordingly, in some embodiments, a PRIME editing system is used to target an uORF located 5′ to a translation start site for the primary ORF, and the use of this system results in a deletion, substitution, or insertion at the nucleic acid encoding the uORF translation start site. This modification of the uORF translation start site results in the decreased translation of the uORF, which thereby increases the translation of the pORF, and improves an agronomic trait of interest encoded by the pORF. Alternatively, in some embodiments, a PRIME editing system is used to target a sequence located 5′ to a translation start site for the primary ORF, and the use of this system results in the insertion of at least one uORF translation start site. This insertion of the uORF translation start site results in the increased translation of the uORF, which thereby decreases the translation of the pORF, and improves an agronomic trait of interest.

### Zinc Finger Nucleases

Another available method for modifying sequences is the use of zinc finger nucleases (ZFN). A ZFN is an artificial restriction enzyme prepared by fusing a zinc finger DNA binding domain with a DNA cleavage domain. The zinc finger DNA binding domain of a single ZFN typically contains 3-6 individual zinc finger repeats, each zinc finger repeat recognizing, for example, 3 bp. ZFN systems suitable for use in the present invention can be obtained, for example, from Shukla et al. (2009) Nature, 459: 437-441; and Townsend et al. (2009) Nature, 459: 442-445.

Accordingly, in some embodiments, a ZFN system is used to target an uORF located 5′ to a translation start site for the primary ORF, and the use of this system results in a deletion, substitution, or insertion at the nucleic acid encoding the uORF translation start site. This modification of the uORF translation start site results in the decreased translation of the uORF, which thereby increases the translation of the pORF, and improves an agronomic trait of interest encoded by the pORF.

### Transactivator-Like Effector Nucleases

“Transactivator-like effector nucleases (TALENs)” are restriction enzymes that can be engineered to cleave specific DNA sequences, usually prepared by fusion of the DNA binding domain of the transcriptional activator-like effector (TALE) and a DNA cleavage domain. TALE can be engineered to bind almost any desired DNA sequences. The TALEN system suitable for use in the present invention can be obtained, for example, from Li et al. (2012) Nat. Biotechnol. 30: 390-392.

Accordingly, in some embodiments, a TALEN system is used to target an uORF located 5′ to a translation start site for the primary ORF, and the use of this system results in a deletion, substitution, or insertion at the nucleic acid encoding the uORF translation start site. This modification of the uORF translation start site results in the decreased translation of the uORF, which thereby increases the translation of the pORF, and improves an agronomic trait of interest encoded by the pORF.

### Higher Protein Levels Improving Agronomic Traits

Potential target uORFs may be those adjacent to pORFs that produce proteins known to be involved in desirable agronomic traits and/or uORFs that are conserved across a group of plant taxa at the species, genus, family, order, or other cladistic level. It is known in the art that many plant genes that encode protein kinases and transcription factors harbor uORFs, and some uORFs regulate crucial developmental processes (von Arnim A. (2014) Plant Sci. 214: 1-12). The effect of putative uORFs may be tested through many probing and assay techniques well known in the art, such as reporter assays. Translation of a reporter gene can be detected in vitro or in vivo. In detection assays, the reporter gene can include a detectable label, such as a fluorescent, radioisotope or chemiluminescent label, or an enzyme label, such as horseradish peroxidase, alkaline phosphatase, or luciferase. Such experimental configurations are laid out in, for example, JP6081412B2.

For further example, a 5′ UTR fragment of the target gene is modified, and a dual-luciferase screening assay is prepared. If the reporter gene construct containing the 5′ UTR of the target gene displays altered fluorescence in the presence of the modified putative uORF, it is then concluded that the inhibition of the 5′UTR fragment of the target gene is at least partially responsible for the modulation in reporter gene expression, making it likely to be an inhibitory uORF. Specific methods for isolating and transfecting protoplasts vary according to species but are known in the art. After delivery of gene editing vectors to plant cell cultures or other plant cell populations, desired uORF mutants may be generated and identified. Protein levels of target genes can then be measured with common laboratory techniques such as qRT-PCR, western blotting, ELISA, immunoprecipitation assays, or reporter assays. The altering of protein levels can validate the effects of the uorfs. In some embodiments, uORFs of the present disclosure exhibit higher levels of reporter gene expression when modified through gene editing. In some embodiments, uORFs of the present disclosure exhibit lower levels of reporter gene expression when modified through gene editing.

Bioinformatic tools can assist in the identification of putative proteins that may benefit plant agronomic traits in an overexpressed or otherwise upregulated state. There are extensive databases available for predicting or otherwise identifying these proteins, such as those built on microarray data, RNA-seq results, general genomic and transcriptomic functional annotation, and metabolomics. Various bioinformatic techniques continue to evolve that assist in this predictive work as well, such as improving genome assembly capabilities. A list of plant bioinformatic databases accessible through open access is available, for example, in Gomez-Casati et al. (2018) Curr. Issues Mol. Biol. 27: 89-104.

Accordingly, in some embodiments, low levels of a pORF protein's expression are associated with a less desirable plant phenotype or less desirable agronomic trait. As used herein, the terms “phenotype” and “characteristic” are used interchangeably and refer to the expression of a gene or genotype, In a non-limiting example, protein levels in a plant displaying this less desirable trait are between about 10% and 100% lower than levels seen in a wild-type plant or a corresponding plant with desirable phenotype, for example, from 10% to 15%, 10% to 20%, 10% to 25%, 10% to 30%, 10% to 35%, 10% to 40%, 10% to 45%, 10% to 50%, 10% to 55%, 10% to 60%, 10% to 65%, 10% to 70%, 10% to 75%, 10% to 80%, 10% to 85%, 10% to 90%, 10% to 95%, and up to about 100% lower than levels seen in a wild-type plant or a corresponding plant with a desirable phenotype. In some embodiments, a corresponding plant with a desirable phenotype is similar to the plant to be edited but does not comprise a translation start site for a uORF 5′ to the translation start site of a pORF. In some embodiments, “low” levels are an objective or comparative measurement, quantitative or qualitative. In some embodiments, high levels of a target protein's expression are associated with a more desirable plant phenotype or more desirable agronomic trait. In a non-limiting example, protein levels in a plant displaying this more desirable trait are between about 10% to 500% higher than levels seen in a wild-type plant or a plant with a corresponding less desirable phenotype, for example, from 10% to 15%, 10% to 20%, 10% to 25%, 10% to 30%, 10% to 35%, 10% to 40%, 10% to 45%, 10% to 50%, 10% to 55%, 10% to 60%, 10% to 65%, 10% to 70%, 10% to 75%, 10% to 80%, 10% to 85%, 10% to 90%, 10% to 95%, 10% to 100%, 10% to 125%, 10% to 150%, 10% to 175%, 10% to 200%, 10% to 225%, 10% to 250%, 10% to 275%, 10% to 300%, up to 325%, up to 350%, up to 375%, up to 400%, up to 425%, up to 450%, up to 475%, and up to about 500% higher than levels seen in a wild-type plant or a plant with a corresponding less desirable phenotype. In some embodiments, “high” levels are an objective or comparative measurement, quantitative or qualitative.

uORFs editing can affect a wide range of agronomic traits. As used herein, the term “agronomic trait” refers to traits deemed beneficial to crop plants. Agronomic traits may refer to traits applicable in plant growth, crop maintenance, and/or traits applicable to the consumption or utilization of the crop plant. Altering a crop plant's genes may be desirable either because genetic regulation in a plant's wild evolution does not align with desired traits for the artificial selection of cultivation, or because genetic editing enables the opportunity to amplify desirable traits beyond the genetic targets or genetic ranges available through traditional plant breeding methods.

Accordingly, some embodiments of the present disclosure include increasing the translation level of pORFs that correspond to desirable agronomic traits. In some embodiments, the primary ORF encodes a protein involved in drought tolerance, disease resistance, pest resistance, stress tolerance, yield, shape, odor, texture, metabolite production, nutrient absorption, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception. In some embodiments, the primary pORF encodes a protein that exhibits at least one less desirable characteristic associated with drought tolerance, disease resistance, pest resistance, stress tolerance, yield, shape, odor, texture, metabolite production, nutrient absorption, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception. In some embodiments, the agronomic trait includes drought tolerance, disease resistance, pest resistance, stress tolerance, yield, shape, odor, texture, metabolite production, nutrient absorption, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception. As used herein, the term “pest resistance” refers to resistance to insect, nematode, fungal disease, and bacterial disease resistance. As used herein, the term “disease resistance” refers to plants avoiding the harmful symptoms that are the outcome of the plant-pathogen interactions. Alongside improving or maintaining the health of a crop germplasm, traits that improve the growth or maintenance of crop plants may provide the plant additional energy for producing the consumptive product of the specific crop. For example, improved branching may allow a fruiting crop better access to sunlight, which in turn allows more energy capture that supplies metabolic material for fruit production.

Investment in improving agronomic traits in crop plants span the financial, environmental, and human health benefits that accompany improved crops. In some embodiments, plants of the present disclosure with improved agronomic traits are more resilient to damage, making monetary investment in growing them more efficient. In some embodiments, the plants with improved agronomic traits are more stable in the face of environmental change. In some embodiments, the plants with improved agronomic traits help stabilize critical food webs. In some embodiments, the plants with improved agronomic traits offer better nutritional value to human consumption or animal feed products, and/or they may help address food security concerns. Accordingly, in some embodiments, provided herein is an edited plant produced by the methods of the present disclosure. Some embodiments cover an edited plant that possesses improved agronomic traits through an uORF's decreased translation. In some embodiments, provided herein is an edited plant that possess improved agronomic traits through a pORF's increased translation.

In some embodiments, the plant or part thereof is a crop plant. In some embodiments, the plant or part thereof includes a leaf, a shoot, a meristem, a stem, or a root. In some embodiments, the plant or part thereof is a monocot. In some embodiments, the plant or plant thereof is selected from the group consisting of maize, rice, sorghum, rye, barley, wheat, sorghum, pearl millet, foxtail millet, oats, sugarcane, turfgrass, and switchgrass. In some embodiments, the plant or part thereof is a dicot. In some embodiments, the plant or plant thereof is selected from the group consisting of flax, canola, soybean, alfalfa, sunflower, cotton, tobacco, peanut, potato, cannabis, tomato, and Arabidopsis. In some embodiments, the plant or plant thereof is selected from the group consisting of a forage crop, an industrial crop, a woody crop, and a biomass crop. As used herein, the term “plant” includes a whole plant and any descendant, cell, tissue, or part of a plant. Any part(s) of a plant include, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks.

### Plant Breeding Methods

The improvement of agronomic traits in a crop plant is a method of improving germplasm or general crop populations, and it results in an elite crop plant. As used herein, the phrase “elite crop plant” refers to a plant which has undergone breeding to provide one or more trait improvements. Elite crop plant lines include plants which are an essentially homozygous, e.g. inbred or doubled haploid. Elite crop plants can include inbred lines used as is or used as pollen donors or pollen recipients in hybrid seed production (e.g. used to produce F1 plants). Elite crop plants can include inbred lines which are selfed to produce non-hybrid cultivars or varieties or to produce (e.g., bulk up) pollen donor or recipient lines for hybrid seed production. Elite crop plants can include hybrid F1 progeny of a cross between two distinct elite inbred or doubled haploid plant lines. In addition, the crossing of distinct elite inbred or doubled haploid plant lines can result in the generation of transgene-free progeny. As used herein, “transgene-free progeny” refers to the progeny of a gene-edited plant(s) that exhibits a segregated-out transgene element. In some embodiments, high levels of protein produced by the uORF are associated with an agronomically undesirable phenotype. In some embodiments, high levels of protein produced by the primary ORF are associated with an agronomically desirable trait. In some embodiments, the gene editing system is provided to the plant by application of a composition including the gene editing system to the plant or part thereof.

Such plant breeding methods are useful at least insofar as they allow for production of distinct useful donor plant lines each having unique sets of modified transgenic loci and, in some instances, targeted genetic changes that are tailored for distinct agronomic traits targeted for improvement. Such elite crop plants can be inbred plant lines or can be hybrid plant lines. In some embodiments, at least one added or modified uORF is introgressed into a desired donor line comprising elite crop plant germplasm. Backcrosses can be repeated and/or supplemented by molecular assisted breeding techniques using SNP or other nucleic acid markers to select for recurrent parent germplasm until a desired recurrent parent percentage is obtained (e.g., at least about 95%, 96%, 97%, 98%, or 99% recurrent parent percentage).

Gene Editing to Add uORFs

While disabling the uORF lowers the level with which ribosomes, or a cell's translational machinery, initiates translation with the uORF, addition one or multiple uORFs can increase the level with which ribosomes, or a cell's translation machinery, initiate translation with the uORF. While decreasing translation of the uORF can increase the translation of the pORF, increasing translation of the uORF can decrease the translation of the pORF. Increased translation of the uORF by more initiation at uORF translation start sites can lower the rate at which ribosomes can re-initiate at the pORF translation start site. In some embodiments, introduction of a uORF modulates expression of a pORF.

In some embodiments, these gene editing techniques are used to insert a translation start site into the 5′ UTR of a target gene. In some embodiments, these gene editing techniques are used to insert an uORF into the 5′ UTR of a target gene. In some embodiments, these gene editing techniques are used to modify sequences near an existing uORF. In some embodiments, the modified sequence is near an existing uORF. In some embodiments, the modified sequence is adjacent to an existing uORF. In some embodiments, the modified sequence is not near an existing uORF. In some embodiments, the modified sequence results in a series of similar or identical uORFs. In some embodiments, the modified sequence results in the insertion of an uORF into sequences without nearby uORFs. In some embodiments, the modified sequence may include the insertion of a heterologous uORF. As used herein, the term “heterologous” refers to any polynucleotide (e.g. DNA molecule) that has been inserted into a new location in the genome of a plant. Non-limiting examples of an exogenous or heterologous DNA molecule include a synthetic DNA molecule, a non-naturally occurring DNA molecule, a DNA molecule found in another species, a DNA molecule found in a different location in the same species, and/or a DNA molecule found in the same strain or isolate of a species, where the DNA molecule has been inserted into a new location in the genome of a plant. In some embodiments, the intention of inserting the uORF is to increase translation rates of the uORF. In some embodiments, the uORF is added to improve agronomic traits by increasing translation rates of the uORF. In some embodiments, the uORF is added to improve agronomic traits by increasing the inhibition of the associated pORF(s). In some embodiments, the uORF is added to improve agronomic traits by decreasing the translation of the associated pORF(s). In some embodiments, the uORF is added to improve agronomic traits by decreasing the protein levels of the associated pORF(s). In some embodiments, inducing change in the uORF sequence increases the rate of uORF translation and decreases the rate of pORF translation, thereby decreasing the level of protein produced by the pORF and improving an agronomic trait.

In some embodiments, the addition of uORFs is accomplished through use of a CRISPR/Cas system. In some embodiments, the Cas associated with the CRISPR/Cas system is a Cas nickase. In some embodiments, the Cas endonuclease is selected from the group consisting of Cas9, Cas12a (Cpf1), Cas12e (CasX), Cas12d (CasY), C2c1, C2c2, C2c3, Cas12h, Cas12i, Cas12j, Cas14, and an engineered Cas nuclease. In some embodiments, the Cas nickase includes a mutation in one or more nuclease active sites. In some embodiments, the Cas nickase is associated with a reverse transcriptase. In some embodiments, the Cas nickase is fused to the reverse transcriptase. In embodiments of the present disclosure, at least one uORF is added to improve agronomic traits through gene editing techniques. In some embodiments, guide RNA binds to a target sequence within a target site that is adjacent to an uORF, nearby an uORF, or creates an uORF. In some embodiments, the guide RNA and/or the endonuclease are provided to the plant by transforming the plant with a vector including nucleic acid encoding the guide RNA and nucleic acid encoding the endonuclease. In some embodiments, the composition including the guide RNA includes a nuclease inhibitor. In some embodiments, the nuclease inhibitor includes an RNase inhibitor. In some embodiments, the guide RNA of the gene editing system includes at its 3′ end a priming site and an edit to be incorporated into the genomic target. As used herein, the term “priming site” refers to a sequence complementary to the 3′ end of the target DNA cut site. In some embodiments, the guide RNA includes a spacer sequence that is complementary to the target sequence and a PAM sequence. In some embodiments, the PAM sequence is located 1 to 30 nucleotides 5′ of the spacer sequence, such as within 1 to 5 nucleotides, 1 to 10 nucleotides, 1 to 15 nucleotides, 1 to 20 nucleotides, 1 to 25 nucleotides, or 1 to 30 nucleotides. In some embodiments, the PAM sequence includes the nucleotide sequence TT, TTT, TTAT, TTTN, TTGT, CTT, TTC, CC, NGG, or a T- or C-rich sequence, wherein the nucleotide N represents any nucleobase. In some embodiments, the PAM sequence includes the nucleotide sequence TTTN. In some embodiments, the priming site that the gene editing targets is within the 5′ UTR of a eukaryote. In some embodiments, the site is within the 5′ UTR of a plant. In some embodiments, the site is within the 5′ UTR of a crop plant.

### Homology-Directed Repair

The insertion of desired sequences or deletion of an uORF may also be accomplished through utilizing homology-directed repair (HDR). HDR is a genome editing method that can be used for precise replacement of a target genomic DNA site with the sequence from a provided DNA template containing the desired replacement sequence. HDR involves the supply of a donor template, or a sequence having homology to the target editing site for uORF insertion or deletion. Accordingly, in some embodiments, the use of a donor template results in the addition of at least one uORF. In some embodiments, the use of a donor template results in the addition of at least one new translation start site. In some embodiments, the use of a donor template results in the deletion of an uORF. As used herein, the terms “donor template” and “donor template nucleic acid” are used interchangeably.

Donor template DNA molecules used in the methods, systems, eukaryotic cells (e.g., plant cells), and compositions provided herein include DNA molecules comprising, from 5′ to 3′, a first homology arm, at least one replacement DNA, and a second homology arm, wherein the homology arms containing sequences that are partially or completely homologous to genomic DNA (gDNA) sequences flanking an endonuclease recognition sequence in the gDNA and wherein the replacement DNA can comprise an insertion, deletion, or substitution of 1 or more DNA base pairs relative to the target gDNA. In certain embodiments, a donor DNA template homology arm is about 20, 50, 100, 200, 400, or 600 to about 800, or 1000 base pairs in length. In certain embodiments, a donor template DNA molecule is delivered to a eukaryotic cell (e.g., a plant cell) in a circular (e.g., a plasmid or a viral vector including a geminivirus vector) or a linear DNA molecule. In certain embodiments, a circular or linear DNA molecule that is used can comprise a modified donor template DNA molecule comprising, from 5′ to 3′, a first copy of an endonuclease recognition sequence, the first homology arm, the replacement DNA, the second homology arm, and a second copy of the endonuclease recognition sequence. In some embodiments, an RNA template of a reverse transcriptase is provided. In some embodiments, a revise transcriptase is provided in addition to an RNA. In some embodiments, the method comprises use of a single stranded DNA donor template. In some a single or double stranded RNA template is used. In some embodiments, the method comprises use of a DNA/RNA hybrid.

Accordingly, in some embodiments, HDR is used to target an uORF located 5′ to a translation start site for the primary ORF, and the use of HDR results in a deletion, substitution, or insertion at the nucleic acid encoding the uORF translation start site. This modification of the uORF translation start site results in the decreased translation of the uORF, which thereby increases the translation of the pORF, and improves an agronomic trait of interest encoded by the pORF. Alternatively, in some embodiments, HDR is used to target a sequence located 5′ to a translation start site for the primary ORF, and the use of HDR results in the insertion of at least one uORF translation start site. This insertion of the uORF translation start site results in the increased translation of the uORF, which thereby decreases the translation of the pORF, and improves an agronomic trait of interest.

### Decreasing Protein/Translation Levels in Plants

In some embodiments the target uORF is adjacent to pORFs that produces a protein known to be involved in less desirable agronomic traits.

Potential target uORFs may be those adjacent to pORFs that produce proteins known to be involved in inhibiting desirable agronomic traits and/or uORFs that are conserved across a group of plant taxa at the species, genus, family, order, or other cladistic level. Accordingly, in some embodiments, low levels of a target protein's expression are associated with a more desirable plant phenotype or more desirable agronomic trait. In a non-limiting example, protein levels in a plant displaying this more desirable trait are 10% to 15%, 10% to 20%, 10% to 25%, 10% to 30%, 10% to 35%, 10% to 40%, 10% to 45%, 10% to 50%, 10% to 55%, 10% to 60%, 10% to 65%, 10% to 70%, 10% to 75%, 10% to 80%, 10% to 85%, 10% to 90%, 10% to 95%, and up to about 100% lower than levels seen in a wild-type plant or a plant with a corresponding less desirable phenotype. “Low” levels may be an objective or comparative measurement, quantitative or qualitative. In some embodiments, high levels of a target protein's expression are associated with a less desirable plant phenotype or less desirable agronomic trait. In a non-limiting example, protein levels in a plant displaying this less desirable trait are 10% to 15%, 10% to 20%, 10% to 25%, 10% to 30%, 10% to 35%, 10% to 40%, 10% to 45%, 10% to 50%, 10% to 55%, 10% to 60%, 10% to 65%, 10% to 70%, 10% to 75%, 10% to 80%, 10% to 85%, 10% to 90%, 10% to 95%, 10% to 100%, 10% to 125%, 10% to 150%, 10% to 175%, 10% to 200%, 10% to 225%, 10% to 250%, 10% to 275%, 10% to 300%, up to 325%, up to 350%, up to 375%, up to 400%, up to 425%, up to 450%, up to 475%, and up to about 500% higher than levels seen in a wild-type plant or a plant with a corresponding more desirable phenotype. “High” levels may be an objective or comparative measurement, quantitative or qualitative.

In some embodiments, the target uORF is conserved across a group of plant taxa at the species, genus, family, order, or other cladistic level.

The term “conserved sequence”, as used herein, means a set of amino acids or nucleotide bases conserved at specific positions along an aligned sequence of evolutionarily related proteins or nucleic acids. While amino acids or nucleic acids at other positions can vary between homologous proteins or homologous nucleic acids, amino acids or nucleotide bases that are highly conserved at specific positions typically indicate amino acids or bases that are essential to the structure, the stability, or the activity of a protein or gene.

In some embodiments, the target uORF's associated pORF is conserved across a group of plant taxa at the species, genus, family, order, or other cladistic level. A “conserved” uORF or pORF sequence is a sequence that is identical or substantially identical across a set of sequences, a set of organisms, or a set of taxa. Accordingly, in some nonlimiting examples, a “conserved” sequence can be a nucleotide or polypeptide sequence sharing at least about 50%, at least about 60%, at least about 70%, at least about 80%, at least about 90%, and up to about 100% similarity between at least about 70%, at least about 80%, at least about 90%, and up to about 100% of compared taxa, compared individuals, or other compared groups. Modeling sequence similarity and shared sequence identity is known to one skilled in the art and may be performed through readily available tools, such as through the National Center for Biotechnology Information's BLAST system (available at blast[dot]ncbi[dot]nlm[dot]nih[dot]gov/Blast[dot]cgi). Comparable levels of similarity are seen throughout the art; as an example of a study employing such levels, see Crowe et al. (2006) BMC Genomics, 7:16. Sequences that are “conserved” at the uORF or pORF level are often highly similar or identical due to shared evolutionary inheritance; the perpetuation of the identical sequences is often due to one or more than one of the following causes: strong selective pressures, structural limitation with few alternative sequences possible, and importance in critical cellular processes. In some embodiments, the targeted uORF's associated pORF is highly conserved across plants for regulating cell division. In some embodiments, the targeted uORF's associated pORF is highly conserved across plants for producing a plant hormone. Some “conserved” sequences, while not originating from the same evolutionary events, are shared between a set of sequences, organisms, or taxa due to convergent evolution, in which the similarity may be due to high functional importance or a limited variation of sequences available, leading to sequences shared by chance or constraint.

In some embodiments, a 5′ UTR is edited to result in insertion of at least one nucleotide, a deletion of at least one nucleotide, a substitution of at least one nucleotide (such as creation of a point mutation in the locus). In some embodiments, the edit, modifies the translation frequency or level of the uORF. In some embodiments, the edit increases the frequency with which the uORF is translated by the native translational machinery of the plant cell(s). In some embodiments, translation of the pORF is fully inhibited. In some embodiments, translation of the pORF is partially inhibited. The pORF's decreased translation results in lower levels of resultant pORF proteins.

In some embodiments, a CRISPR/Cas system is used to introduce at least one uORF start codon that decreases pORF translation. In some embodiments, editing by the endonuclease and the guide RNA results in increased translation of the at least one uORF. In some embodiments, protein level of the protein encoded by the primary ORF is decreased. In some embodiments, the uORF protein level is increased by about 30% to 500%, for example, between 30% and 40%, 30% and 50%, 30% and 60%, 30% and 70%, 30% and 75%, 30% and 80%, 30% and 85%, 30% and 90%, 30% and 95%, 30% and 100%, 30% and 125%, 30% and 150%, 30% and 175%, 30% and 200%, 30% and 225%, 30% and 250%, 30% and 275%, 30% and 300%, 30% and 325%, 30% and 350%, 30% and 375%, 30% and 400%, 30% and 425%, 30% and 450%, 30% and 475%, or 30% and up to about 500%. In some embodiments, the pORF protein level is decreased by 30% to about 100%, for example, between 30% and 40%, 30% and 50%, 30% and 60%, 30% and 70%, 30% and 75%, 30% and 80%, 30% and 85%, 30% and 90%, 30% and 95%, and up to about 100%. Target protein levels can be measured through methods known to one skilled in the art, such as Western blotting, immunoprecipitation assays, enzyme-linked immunosorbent assays (ELISA), and other tests. In some embodiments, uORF protein level is increased relative to the level prior to introducing the uORF start codon. In some embodiments, the pORF protein level is decreased relative to the pORF level prior to introducing the uORF start codon.

One aspect of the present disclosure includes a method of improving an agronomic trait in a plant comprising providing a gene editing system to the plant or part thereof, wherein the gene editing system edits a target site, wherein the target site comprises nucleic acid encoding a translation start site for an upstream open reading frame (uORF) located 5′ to a translation start site for a primary open reading frame (pORF), wherein editing by the gene editing system introduces a translation start site for an upstream open reading frame (uORF) in the target site. Another aspect of the present disclosure includes a method of improving an agronomic trait in a plant including providing an endonuclease and a guide RNA for the endonuclease to the plant or part thereof, wherein the guide RNA is complementary to a target sequence in a target site, wherein the target site is located 5′ to a translation start site for a primary open reading frame (ORF), wherein editing by the guide RNA and the endonuclease introduces a translation start site for an upstream open reading frame (uORF) in the target site. One aspect of the present disclosure includes a method of decreasing translation of a primary open reading frame (ORF) in a plant comprising providing a gene editing system, wherein the gene editing system edits a target site, wherein the target site comprises nucleic acid encoding a translation start site for an upstream open reading frame (uORF) located 5′ to a translation start site for a primary open reading frame (pORF), wherein editing by the gene editing system introduces a translation start site for an upstream open reading frame (uORF) in the target site. Another aspect of the present disclosure includes a method of decreasing translation of a primary open reading frame (ORF) in a plant including providing an endonuclease and a guide RNA for the endonuclease to the plant, wherein the guide RNA is complementary to a target sequence in a target site, wherein the target site is located 5′ to a translation start site for the primary ORF, wherein editing by the guide RNA and the endonuclease introduces a translation start site for an upstream open reading frame (uORF) in the target site. In some embodiments, editing by the gene editing system results in translation of a peptide or polypeptide from the new translation start site. In some embodiments, editing by the endonuclease and the guide RNA results in translation of a peptide or polypeptide from the new translation start site. In some embodiments, the new translation start site is 1 to about 500 base pairs from the translation start site of the pORF, for example, 1 to 50 base pairs, 1 to 100 base pairs, 1 to 150 base pairs, 1 to 200 base pairs, 1 to 250 base pairs, 1 to 300 base pairs, 1 to 350 base pairs, 1 to 400 base pairs, 1 to 450 base pairs, or 1 to about 500 base pairs. In some embodiments, the polypeptide that results from the new translation start site is between about 1 and 230 amino acids in length, for example about 1 to 6 amino acids, 1 to 15 amino acids, 1 to 20 amino acids, 1 to 30 amino acids, 1 to 40 amino acids, 1 to 50 amino acids, 1 to 60 amino acids, 1 to 75 amino acids, 1 to 80 amino acids, 1 to 90 amino acids, 1 to 100 amino acids, 1 to 120 amino acids, 1 to 135 amino acids, 1 to 150 amino acids, 1 to 165 amino acids, 1 to 175 amino acids, 1 to 190 amino acids, 1 to 200 amino acids, 1 to 215 amino acids, and up to about 1 to 230 amino acids in length.

In some embodiments, a PRIME editing system is used to target a sequence located 5′ to a translation start site for the primary ORF, and the use of the PRIME editing system results in the insertion of at least one uORF translation start site. The formation of a new translation start site may result from the deletion, substitution, or insertion of at least one nucleotide at the target site. This insertion of the uORF translation start site results in the increased translation of the uORF, which thereby decreases the translation of the pORF, and improves an agronomic trait of interest.

In some embodiments, a Precise Base Editing (PBE) system is used to target a sequence located 5′ to a translation start site for the primary ORF, and the use of the PBE system results in the insertion of at least one uORF translation start site. This insertion of the uORF translation start site results in the increased translation of the uORF, which thereby decreases the translation of the pORF, and improves an agronomic trait of interest.

In some embodiments, a zinc finger nuclease (ZFN) system is used to target a sequence located 5′ to a translation start site for the primary ORF, and the use of the ZFN system results in the insertion of at least one uORF translation start site. This insertion of the uORF translation start site results in the increased translation of the uORF, which thereby decreases the translation of the pORF, and improves an agronomic trait of interest.

In some embodiments, a transactivator-like effector nuclease (TALEN) system is used to target a sequence located 5′ to a translation start site for the primary ORF, and the use of the TALEN system results in the insertion of at least one uORF translation start site. This insertion of the uORF translation start site results in the increased translation of the uORF, which thereby decreases the translation of the pORF, and improves an agronomic trait of interest.

### Lower Protein Levels Improving Agronomic Traits

Accordingly, in some embodiments, the new translation start site is one selected from the group consisting of AUG, ACG, CUG, UUG, AUA, and AUC. In some embodiments, the new translation site comprises the bases AUG. The various possible selections of a start site for insertion can lead to varying level of uORF translation. The selection of an uORF start site can thereby also affect the level of pORF translation.

Accordingly, in some embodiments, low levels of a target protein's expression are associated with a more desirable plant phenotype or more desirable agronomic trait. In a non-limiting example, protein levels in a plant displaying this more desirable trait may be 10%, 15%, 20%, 25%, 30%, 35%, 40%, 45%, 50%, 55%, 60%, 65%, 70%, 75%, 80%, 85%, 90%, 95%, and up to 100% lower than levels seen in a wild-type plant or a plant with a corresponding less desirable phenotype. “Low” levels may be an objective or comparative measurement, quantitative or qualitative. In some embodiments, high levels of a target protein's expression are associated with a less desirable plant phenotype or less desirable agronomic trait. In a non-limiting example, protein levels in a plant displaying this less desirable trait may be 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, and up to 500% higher than levels seen in a wild-type plant or a plant with a corresponding more desirable phenotype. “High” levels may be an objective or comparative measurement, quantitative or qualitative.

The targeted addition of uORFs can affect a wide range of agronomic traits. As used herein, the term “agronomic trait” refers to traits deemed beneficial to crop plants. Agronomic traits may refer to traits applicable in plant growth, crop maintenance, and/or traits applicable to the consumption or utilization of the crop plant. Altering a crop plant's genes may be desirable either because genetic regulation in a plant's wild evolution does not align with desired traits for the artificial selection of cultivation, or because genetic editing enables the opportunity to amplify desirable traits beyond the genetic targets or genetic ranges available through traditional plant breeding methods.

Accordingly, some embodiments of the present disclosure include decreasing the translation level of pORFs that correspond to less desirable agronomic traits. In some embodiments, the primary pORF encodes a protein involved in drought tolerance, disease resistance, pest resistance, stress tolerance, yield, shape, odor, texture, metabolite production, nutrient absorption, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception. In some embodiments, the agronomic trait includes drought tolerance, disease resistance, pest resistance, stress tolerance, yield, shape, odor, texture, metabolite production, nutrient absorption, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception. In some embodiments, the primary pORF encodes a protein that exhibits at least one less desirable characteristic associated with drought tolerance, disease resistance, pest resistance, stress tolerance, yield, shape, odor, texture, metabolite production, nutrient absorption, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception. In some embodiments, the agronomic trait includes drought tolerance, disease resistance, pest resistance, stress tolerance, yield, shape, odor, texture, metabolite production, nutrient absorption, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception. Alongside improving or maintaining the health of a crop germplasm, traits that improve the growth or maintenance of crop plants may provide the plant additional energy for producing the consumptive product of the specific crop. For example, improved branching may allow a fruiting crop better access to sunlight, which in turn allows more energy capture that supplies metabolic material for fruit production.

In some embodiments, plants of the present disclosure with improved agronomic traits are more resilient to damage, making monetary investment in growing them more efficient. In some embodiments, the plants with improved agronomic traits are more stable in the face of environmental change. In some embodiments, the plants with improved agronomic traits help stabilize critical food webs. In some embodiments, the plants with improved agronomic traits offer better nutritional value to human consumption or animal feed products, and/or they may help address food security concerns. Accordingly, some embodiments cover an edited plant produced by the methods of the present disclosure. Some embodiments cover an edited plant that possesses improved agronomic traits through an uORF's increased translation. Some embodiments cover an edited plant that possess improved agronomic traits through a pORF's decreased translation.

In some embodiments, the plant or part thereof is a crop plant. In some embodiments, the plant or part thereof includes a leaf, a shoot, a meristem, a stem, or a root. In some embodiments, the plant or part thereof is a monocot. In some embodiments, the plant or plant thereof is selected from the group consisting of maize, rice, sorghum, rye, barley, wheat, sorghum, pearl millet, foxtail millet, oats, sugarcane, turfgrass, and switchgrass. In some embodiments, the plant or part thereof is a dicot. In some embodiments, the plant or plant thereof is selected from the group consisting of flax, canola, soybean, alfalfa, sunflower, cotton, tobacco, peanut, potato, cannabis, tomato, and Arabidopsis. In some embodiments, the plant or plant thereof is selected from the group consisting of a forage crop, an industrial crop, a woody crop, and a biomass crop. As used herein, the term “plant” includes a whole plant and any descendant, cell, tissue, or part of a plant. Any part(s) of a plant include, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks. In some embodiments, high levels of protein produced by the pORF are associated with an agronomically undesirable phenotype. In some embodiments, high levels of protein produced by the uORF are associated with an agronomically desirable trait.

### Transformation in Plants

Gene editing may be performed through plant transformation techniques, to produce a DSB and/or to introduce an expression vector for the gene editing system into a plant. As used herein, the term “introduction” or “transformation” as refers to the transfer of an exogenous polynucleotide into a host cell, irrespective of the method used for transfer. As used herein, the terms “plasmid”, “vector” and “cassette” refer to an extra-chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art.

In some embodiments, the gene editing system is introduced via particle bombardment/biolistic transformation or Agrobacterium transformation of a recombinant DNA construct comprising the corresponding gene editing system operably linked to a plant promoter. In some embodiments, the guide RNA is introduced via particle bombardment/biolistic transformation or Agrobacterium transformation of a recombinant DNA construct comprising the corresponding guide DNA operably linked to a plant promoter. In some embodiments, the vector used in gene editing is a T-DNA. Transformation methods can thus be utilized to alter sequences in the 5′ UTR of a target gene, to either add or disable uORFs, and this can alter the expression of an agronomic trait.

### Multiplex Editing

In principle, multiplex genetic manipulation means expressing multiple editing gRNAs (or Cas-derived effectors) for corresponding target sites. The use of multiple editing gRNAs can enable the more effective alteration of a given trait, the ability to affect polygenic traits more effectively, or the targeting of multiple traits at once. In some embodiments, uORF editing is used to improve plant agronomic traits by targeting multiple genes at once. In some embodiments, uORF editing improves plant agronomic traits by targeting multiple genes for the same trait. In some embodiments, uORF editing improves plant agronomic traits by targeting multiple genes for multiple traits. In some embodiments, uORF editing improves plant agronomic traits by targeting one gene for multiple traits. In some embodiments, uORF editing improves plant agronomic traits by targeting one gene many times for a single trait. In some embodiments, multiplex editing is used through a CRISPR/Cas system to alter the expression level of at least one uORF and resultant pORF(s).

Accordingly, in some embodiments, improving an agronomic trait includes multiplex editing to target multiple editing points that frame an uORF, subsequently editing out the uORF sequence in a phenomenon referred to as “pop-out” editing. In some embodiments, two or more guide RNAs are encoded by a single precursor RNA. In some embodiments, the two or more guide RNAs are each flanked by a direct repeat. In some embodiments, the method of improving an agronomic trait includes providing two or more, three or more, four or more, or five or more forms of gene editing systems to the plant. In some embodiments, the method of improving an agronomic trait includes providing two or more, three or more, four or more, or five or more rounds of gene editing to the plant. In some embodiments, the method of improving an agronomic trait includes providing two or more, three or more, four or more, or five or more guide RNAs to the plant. In some embodiments, each gene editing system binds a target site, wherein each target site includes nucleic acid encoding a uORF translation start site located 5′ to a translation start site for a primary ORF. In some embodiments, each guide RNA is complementary to a target sequence in a target site, wherein each target site includes nucleic acid encoding a uORF translation start site located 5′ to a translation start site for a primary ORF. In other embodiments, each target site is located 5′ to the translation start site of the same primary ORF. In other embodiments, each target site is located 5′ to the translation start site of different primary ORFs. In other embodiments, two or more, three or more, four or more, or five or more uORFs are introduced into the plant or part thereof. In other embodiments, two or more, three or more, four or more, or five or more uORFs are removed or added. In some embodiments, two or more, three or more, four or more, or five or more translation start sites are introduced into the plant or part thereof.

### Expression Cassettes

Included in the transformations of the present disclosure are vectors that include transformation cassettes and include expression cassettes. “Transformation cassette” refers to a specific vector containing a gene and having elements in addition to the gene that facilitates transformation of a particular host cell. “Expression cassette” refers to a specific vector containing a gene and having elements in addition to the gene that allow for expression of that gene in a host. In some embodiments, the vector or expression cassette includes additional elements for improving delivery to a plant cell or plant protoplast or for directing or modifying expression of one or more gene editing system elements, for example, fusing a sequence encoding a cell-penetrating peptide, localization signal, transit, or targeting peptide to a Cas endonuclease, or adding a nucleotide sequence to stabilize a guide RNA. In some embodiments, multiple expression cassettes are delivered to the plant or plant part thereof.

Common constructions of expression vectors include the linking of a donor template to a promoter that drives expression of the donor template's sequence intended for insertion into the recipient cell. In some embodiments, the nucleic acid encoding the guide RNA and the nucleic acid encoding the endonuclease are operably linked to a promoter. In some embodiments, the nucleic acid encoding the guide RNA and the nucleic acid encoding the endonuclease are operably linked to different promoters. In some embodiments, the promoter is inducible or constitutive. In some embodiments, the promoter is selected from the group consisting of CaMV35S. In some embodiments, the promoter is selected from the group consisting of ubiquitin. In some embodiments, the promoter is selected from the group consisting of CaMV35S, ubiquitin, Rsyn7, NOS, MAS, ALS, pEMU, AtU3, AtU6, OsU3, OsU6, Pol II, Pol III, a tissue-specific promoter, and a cell-specific type promoter.

### Donor Templates

In some embodiments, the delivered gene editing system that alters at least one uORF sequence for improving agronomic traits includes a donor template. In some embodiments, the delivered guide RNA that alters at least one uORF sequence for improving agronomic traits includes a donor template. As used herein, the term “donor template” refers to a DNA construct that includes a polynucleotide of interest to be inserted into the target site of a gene editing system, as well as potentially including a first and a second region of homology that flank the polynucleotide of interest. In some embodiments, the polynucleotide of interest is an uORF to be added to a target sequence in the plant or plant part thereof. In some embodiments, the polynucleotide of interest is a translation start site to be added to a target sequence in the plant or plant part thereof. In some embodiments, modifying a target site in the genome of a plant cell includes introducing a guide RNA and a donor DNA template into a plant cell having a Cas endonuclease, wherein said guide RNA and Cas endonuclease are capable of forming a complex that enables the Cas endonuclease to introduce a DSB at said target site, wherein said donor DNA comprises a polynucleotide of interest.

In some embodiments, the donor template includes a translation start site to insert into a target gene's 5′ UTR. In some embodiments, the donor template includes a uORF to insert into a target gene's 5′ UTR. In some embodiments, the donor template includes at least one uORF to insert into a target gene's 5′ UTR. In some embodiments, the donor template includes duplicate uORFs to insert into a target gene's 5′ UTR. In some embodiments, the modifications of a 5′ UTR include introduction of double stranded breaks followed by non-homologous end joining (NHEJ) either in the presence or absence of a donor DNA template that lacks homology to the site of the double stranded break. In some embodiments, the method of improving agronomic traits and/or altering translation includes providing a donor template nucleic acid. In some embodiments, the donor template nucleic acid is inserted at the target site. In some embodiments, insertion of the donor template nucleic acid results in removal of a uORF translation start site. In some embodiments, insertion of the donor template nucleic acid results in insertion of a new translation start site.

### Delivery and Application of the Gene Editing System

Included in the present disclosure are methods of introducing the gene editing system, sometimes including at least one guide RNA, expression cassette, transformation element, or constructed gene-editing vector to a plant or plant part thereof. In some embodiments, the gene editing system is provided to the plant by application of a composition including the gene editing system to the plant or part thereof. In some embodiments, the guide RNA is provided to the plant by application of a composition including the guide RNA to the plant or part thereof. In other embodiments, the gene editing system is provided to the plant by spraying the plant with the composition including the gene editing system. In other embodiments, the guide RNA is provided to the plant by spraying the plant with the composition including the guide RNA. In other embodiments, the composition including the gene editing system includes a surfactant. In other embodiments, the composition including the gene editing system includes glass beads coating the gene editing system. In other embodiments, application of the gene editing system includes rubbing a composition including the gene editing system onto the leaves, shoot, stem, and/or meristem. In other embodiments, application of the gene editing system includes injecting a composition including the gene editing system into the stem. In other embodiments, the composition including the guide RNA includes a surfactant. In other embodiments, the composition including the guide RNA includes glass beads coating the guide RNA. In other embodiments, application of the guide RNA includes rubbing a composition including the guide RNA onto the leaves, shoot, stem, and/or meristem. In other embodiments, application of the guide RNA includes injecting a composition including the guide RNA into the stem. In other embodiments, the gene editing system is operably linked to a plant promoter. In other embodiments, the guide DNA is operably linked to a plant promoter. In other embodiments, a plant promoter is used to express a Cas system and the guide RNA. In other embodiments, a plant promoter is used to express Cas9 and the guide RNA. Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700).

In some embodiments, application of the guide RNA includes leaf infiltration of a composition including the gene editing system into the leaf. In some embodiments, application of the guide RNA includes leaf infiltration of a composition including the guide RNA into the leaf. In other embodiments, the leaf infiltration includes forced infiltration using a needle-less syringe or vacuum pump. In other embodiments, application includes biolistic transformation of nucleic acid encoding the gene editing system into the leaf, shoot, shoot, stem, and/or meristem. In other embodiments, application includes biolistic transformation of nucleic acid encoding the guide RNA into the leaf, shoot, shoot, stem, and/or meristem. In other embodiments, the biolistic transformation includes transformation of circular DNA encoding the gene editing system. In other embodiments, the biolistic transformation includes transformation of circular DNA encoding the guide RNA. In other embodiments, the method of improving agronomic traits or altering translation includes retrieving a progeny of the plant, where the progeny has an edited target sequence. In other embodiments, progeny is collected from the plants produced by any of the methods included in this disclosure.

Having generally described this invention, the same will be better understood by reference to certain specific examples, which are included herein to further illustrate the invention and are not intended to limit the scope of the invention as defined by the claims.

The following words and phrases have the meanings set forth below.

As used herein, the term “agronomic trait” refers to a characteristic of a plant, which includes, but is not limited to, plant morphology, physiology, growth and development, yield, nutritional enhancement, disease or pest resistance, or environmental or chemical tolerance. “Agronomic traits” are also particularly relevant traits for crop plants. “Agronomic traits” are plant characteristics useful for various forms of ethnobotany and agriculture, directly or indirectly.

As used herein, the term “backcross” refers to crossing an F1 plant or plants with one of the original parents. A backcross is used to maintain or establish the identity of one parent (species) and to incorporate a particular trait from a second parent (species).

As used herein, the phrase “improving an agronomic trait” in a plant refers to either the measurable enhancement, increase, or introduction of a desirable plant trait, or to the measurable downgrading, decrease, or cessation of an undesirable plant trait.

As used herein, the term “isolated” means having been removed from its natural environment.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

As used herein, the phrase “operably linked” refers to a juxtaposition wherein the components so described are in a relationship permitting them to function in their intended manner. For instance, a promoter is operably linked to a coding sequence if the promoter affects its transcription or expression. When the phrase “operably linked” is used in the context of a PAM site and a guide RNA hybridization site, it refers to a PAM site which permits cleavage of at least one strand of DNA in a polynucleotide with an RNA dependent DNA endonuclease or RNA dependent DNA nickase which recognize the PAM site when a guide RNA complementary to guide RNA hybridization site sequences adjacent to the PAM site is present.

As used herein, the term “pORF” refers to a “primary open reading frame”, or the coding sequence of a gene of interest involved an agronomic trait. A pORF includes a start codon and a stop codon, and it may be various lengths and various distances from an associated upstream open reading frame (uORF).

As used herein, the term “priming site” refers to a sequence complementary to the 3′ end of the target DNA cut site.

As used herein, the term “uORF” or “upstream open reading frame” refers to an mRNA element, defined by a start codon in the 5′-untranslated region of an open reading frame of interest. The uORF may possess many qualities, such as being in-frame or out-of-frame, overlapping with the primary open reading frame or fully preceding it, and comprise various lengths.

### EXAMPLES

### Example 1: Upregulating pORF Gene Activity Through Manipulation of uORFs

At least one desirable protein of interest in a plant or plant part is identified through methods known in the art, wherein the at least one protein's activity is responsible for a desirable agronomic trait. Identifying proteins of interest is accomplished through, for example, comparing the level of certain protein between plants exhibiting the desirable agronomic trait and plants not exhibiting the desirable agronomic trait. Functional gene studies and analysis of available genetic databases also allow the identification of such proteins of interest.

After identification of a protein of interest, the primary open reading frame (pORF) which encodes the protein of interest is identified. The sequence upstream of the pORF's translation start site, located 5′ to the pORF translation start site, is then scanned for a start codon and, potentially, a stop codon. This start codon that is upstream of the pORF is identified as a potential regulatory upstream open reading frame (uORF). Further characterization of this upstream sequence as an uORF may be performed through ribosomal profiling, which reports ribosomal occupancy of mRNA, or other methods known in the art.

A gene editing system is delivered to the plant or plant part in order to edit a target site, which here is the translation start site for the uORF.

Editing by the gene editing system results in a deletion, substitution, or insertion at the nucleic acid encoding the uORF translation start site, causing inactivation of the uORF translation start site. The level of protein produced from the uORF is decreased upon successful editing, while the level of protein produced from the pORF is increased.

Validation of the gene editing is conducted according to methods known in the art, such as gene sequencing.

Protein levels are assessed through methods known in the art, such as through Western blotting, immunoprecipitation assays, and/or enzyme-linked immunosorbent assays (ELISA). Increased levels of the protein of interest's activity are observed.

### Example 2: Downregulating pORF Gene Activity Through Manipulation of uORFs

At least one desirable protein of interest in a plant or plant part is identified through methods known in the art, wherein the at least one protein's activity is responsible for a desirable agronomic trait. Identifying proteins of interest is accomplished through, for example, comparing the level of certain protein between plants exhibiting the desirable agronomic trait and plants not exhibiting the desirable agronomic trait. Functional gene studies and analysis of available genetic databases also allow the identification of such proteins of interest.

After identification of a protein of interest, the primary open reading frame (pORF) which encodes the protein of interest is identified.

A gene editing system is delivered to the plant or plant part in order to edit a target site, which here is nucleic acid sequence located 5′ to a translation start site for the pORF. This nucleic acid sequence is suitable for inserting an uORF.

Editing by the gene editing system results in the insertion of nucleic acid encoding at least one uORF translation start site at the target site.

The level of protein produced from the inserted uORF is increased upon successful editing, while the level of protein produced through the pORF is decreased.

Validation of the gene editing is conducted according to methods known in the art, such as gene sequencing.

Protein levels are assessed through methods known in the art, such as through Western blotting, immunoprecipitation assays, and/or enzyme-linked immunosorbent assays (ELISA). Increased levels of the protein of interest's activity are observed.

### Enumerated Embodiments

Embodiment 1. A method of improving an agronomic trait in a plant or part thereof comprising providing a gene editing system to the plant or part thereof, wherein the gene editing system edits a target site, wherein the target site comprises nucleic acid encoding a translation start site for an upstream open reading frame (uORF) located 5′ to a translation start site for a primary open reading frame (pORF), wherein editing by the gene editing system results in a deletion, substitution, or insertion at the nucleic acid encoding the uORF translation start site.

Embodiment 2. A method of increasing translation of a primary open reading frame (pORF) in a plant or part thereof comprising providing a gene editing system to the plant or part thereof, wherein the gene editing system edits a target site, wherein the target site comprises nucleic acid encoding a translation start site for an upstream open reading frame (uORF) located 5′ to a translation start site for the primary ORF, wherein editing by the gene editing system results in a deletion, substitution, or insertion at the nucleic acid encoding the uORF translation start site.

Embodiment 3. The method of any one of embodiments 1-2, wherein editing by the gene editing system results in decreased translation of the uORF.

Embodiment 4. The method of any one of embodiments 1-3, wherein protein level of the protein encoded by the primary ORF is increased.

Embodiment 5. A method of improving an agronomic trait in a plant comprising providing a gene editing system to the plant or part thereof, wherein the gene editing system edits a target site, wherein the target site comprises nucleic acid encoding a translation start site for an upstream open reading frame (uORF) located 5′ to a translation start site for a primary open reading frame (pORF), wherein editing by the gene editing system introduces a translation start site for an upstream open reading frame (uORF) in the target site.

Embodiment 6. A method of decreasing translation of a primary open reading frame (ORF) in a plant comprising providing a gene editing system, wherein the gene editing system edits a target site, wherein the target site comprises nucleic acid encoding a translation start site for an upstream open reading frame (uORF) located 5′ to a translation start site for a primary open reading frame (pORF), wherein editing by the gene editing system introduces a translation start site for an upstream open reading frame (uORF) in the target site.

Embodiment 7. The method of any one of embodiments 5-6, wherein editing by the gene editing system results in increased translation of the uORF.

Embodiment 8. The method of any one of embodiments 5-7, wherein protein level of the protein encoded by the primary ORF is decreased.

Embodiment 9. The method of any one of embodiments 1-8, wherein the gene editing system comprises a precise base editing (PBE) system, a PRIME editing system, a CRISPR-Cas9 system, a CRISPR-Cpf1 system, homing endonucleases, a meganuclease, a zinc finger nuclease system, or a transcription activator-like effector nuclease (TALEN) system.

Embodiment 10. The method of any one of embodiments 1-9, wherein the gene editing system comprises a Cas endonuclease and a guide RNA for the Cas endonuclease.

Embodiment 11. The method of embodiment 5 or 6, wherein editing by the gene editing system results in translation of a peptide or polypeptide from the new translation start site.

Embodiment 12. The method of any one of embodiments 1-11, wherein the primary ORF encodes a protein involved in drought tolerance, disease resistance, pest resistance, stress tolerance, yield, shape, odor, texture, metabolite production, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception.

Embodiment 13. The method of any one of embodiments 1, 3-5, and 7-12, wherein the agronomic trait comprises drought tolerance, disease resistance, pest resistance, stress tolerance, yield, shape, odor, texture, metabolite production, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception.

Embodiment 14. The method of any one of embodiments 1-13, wherein the uORF translation start site is within 500 base pairs of the translation start site for the primary ORF.

Embodiment 15. The method of any one of embodiments 1-14, wherein the uORF translation start site comprises a nucleotide sequence selected from the group consisting of AUG, ACG, CUG, UUG, AUA, and AUC.

Embodiment 16. The method of any one of embodiments 1-15, wherein the primary ORF translation start site comprises a nucleotide sequence selected from the group consisting of AUG, ACG, CUG, UUG, AUA, and AUC.

Embodiment 17. The method of any one of embodiments 1-16, wherein the uORF translation start site is in-frame with the primary ORF translation start site.

Embodiment 18. The method of any one of embodiments 1-16, wherein the uORF translation start site is out-of-frame with the primary ORF translation start site.

Embodiment 19. The method of any one of embodiments 1-18, wherein low or high levels of protein produced by the primary ORF are associated with an agronomically undesirable phenotype.

Embodiment 20. The method of any one of embodiments 1-18, wherein low or high levels of protein produced by the primary ORF are associated with an agronomically desirable phenotype.

Embodiment 21. The method of any one of embodiments 10-20, comprising providing two or more, three or more, four or more, or five or more guide RNAs to the plant as part of the gene editing system.

Embodiment 22. The method of embodiment 21, wherein each guide RNA is complementary to a target sequence in a target site, wherein each target site comprises a nucleic acid encoding a uORF translation start site located 5′ to a translation start site for a primary ORF.

Embodiment 23. The method of embodiment 22, wherein each target site is located 5′ to the translation start site of the same primary ORF.

Embodiment 24. The method of embodiment 22, wherein each target site is located 5′ to the translation start site of different primary ORFs.

Embodiment 25. The method of any one of embodiments 5-24, wherein two or more, three or more, four or more, or five or more uORFs are introduced into the plant or part thereof.

Embodiment 26. The method of any one of embodiments 1-4 and 9-24, wherein two or more, three or more, four or more, or five or more uORFs are removed.

Embodiment 27. The method of any one of embodiments 1-26, wherein the gene editing system comprises a Cas endonuclease is selected from the group consisting of Cas9, Cas12a (Cpf1), Cas12e (CasX), Cas12d (CasY), C2c1, C2c2, C2c3, Cas12h, Cas12i, Cas12j, Cas14, and an engineered Cas nuclease.

Embodiment 28. The method of any one of embodiments 1-27, further comprising providing a donor template nucleic acid.

Embodiment 29. The method of embodiment 28, wherein the donor template nucleic acid is inserted at the target site.

Embodiment 30. The method of embodiment 28 or 29, wherein insertion of the donor template nucleic acid results in removal of a uORF translation start site.

Embodiment 31. The method of embodiment 28 or 29, wherein insertion of the donor template nucleic acid results in insertion of a new translation start site.

Embodiment 32. The method of embodiment 27, wherein the Cas endonuclease is a Cas nickase.

Embodiment 33. The method of embodiment 32, wherein the Cas nickase comprises a mutation in one or more nuclease active sites.

Embodiment 34. The method of embodiment 32 or 33, wherein the Cas nickase is associated with a reverse transcriptase.

Embodiment 35. The method of embodiment 34, wherein the Cas nickase is fused to the reverse transcriptase.

Embodiment 36. The method of any one of embodiments 30-35, wherein the guide RNA comprises at its 3′ end a priming site and an edit to be incorporated into the genomic target.

Embodiment 37. The method of any one of embodiments 10 and 12-36, wherein the guide RNA comprises a spacer sequence that is complementary to the target sequence and a protospacer adjacent motif (PAM) sequence.

Embodiment 38. The method of embodiment 37, wherein the PAM sequence is located 1 to 30 nucleotides 5′ of the spacer sequence.

Embodiment 39. The method of embodiment 38, where the PAM sequence comprises the nucleotide sequence TT, TTT, TTAT, TTTN, TTGT, CTT, TTC, CC, NGG, or a T- or C-rich sequence, wherein the nucleotide N represents any nucleobase.

Embodiment 40. The method of any one of embodiments 1-39, wherein the plant or part thereof is a crop plant.

Embodiment 41. The method of any one of embodiments 1-40, wherein the plant or part thereof is a monocot or a dicot.

Embodiment 42. The method of any one of embodiments 1-41, wherein the plant or part thereof is selected from the group consisting of maize, rice, sorghum, rye, barley, wheat, pearl millet, foxtail millet, flax, oats, sugarcane, turfgrass, switchgrass, soybean, canola, alfalfa, sunflower, cotton, tobacco, tomato, peanut, potato, cannabis, tomato, a forage crop, an industrial crop, a woody crop, a biomass crop, and Arabidopsis.

Embodiment 43. The method of any one of embodiments 1-42, wherein the gene editing system is provided to the plant by transforming the plant with a vector comprising nucleic acid encoding a guide RNA and nucleic acid encoding an endonuclease.

Embodiment 44. The method of embodiment 43, wherein the vector is a T-DNA.

Embodiment 45. The method of any one of embodiments 43-44, wherein the nucleic acid encoding the guide RNA and the nucleic acid encoding the endonuclease are operably linked to a promoter.

Embodiment 46. The method of embodiments 43-45, wherein the nucleic acid encoding the guide RNA and the nucleic acid encoding the endonuclease are operably linked to different promoters.

Embodiment 47. The method of any one of embodiments 45-46, wherein the promoter is inducible or constitutive.

Embodiment 48. The method of any one of embodiments 45-47, wherein the promoter is selected from the group consisting of CaMV35S, ubiquitin, Rsyn7, NOS, MAS, ALS, pEMU, AtU3, AtU6, OsU3, OsU6, Pol II, Pol III, a tissue-specific promoter, and a cell-specific type promoter.

Embodiment 49. The method of any one of embodiments 1-48, wherein the plant or part thereof comprises a leaf, a shoot, a meristem, a stem, or a root.

Embodiment 50. The method of any one of embodiments 1-49, wherein the gene editing system is provided to the plant by application of a composition comprising the gene editing system to the plant or part thereof.

Embodiment 51. The method of embodiment 50, wherein the gene editing system is provided to the plant by spraying the plant with the composition comprising the gene editing system.

Embodiment 52. The method of any one of embodiments 50-51, wherein the composition comprising the gene editing system comprises a surfactant.

Embodiment 53. The method of any one of embodiments 50-52, wherein the composition comprising the gene editing system comprises glass beads coating the guide RNA.

Embodiment 54. The method of any one of embodiments 50 and 52-53, wherein application of the gene editing system comprises rubbing a composition comprising the gene editing system onto the leaves, shoot, stem, and/or meristem.

Embodiment 55. The method of any one of embodiments 50 and 52, wherein application of the gene editing system comprises injecting a composition comprising the gene editing system into the stem.

Embodiment 56. The method of embodiment 50, wherein application of the gene editing system comprises leaf infiltration of a composition comprising the gene editing system into the leaf.

Embodiment 57. The method of embodiment 56, wherein the leaf infiltration comprises forced infiltration using a needle-less syringe or vacuum pump.

Embodiment 58. The method of any one of embodiments 50-57, wherein the composition comprising the gene editing system comprises a nuclease inhibitor.

Embodiment 59. The method of embodiment 58, wherein the nuclease inhibitor comprises an RNase inhibitor.

Embodiment 60. The method of any one of embodiments 50 and 58-59, wherein application comprises biolistic transformation of nucleic acid encoding the gene editing system into a leaf, shoot, shoot, stem, and/or meristem of the plant or part thereof.

Embodiment 61. The method of embodiment 60, wherein the biolistic transformation comprises transformation of circular DNA encoding the gene editing system.

Embodiment 62. The method of any one of embodiments 1-61, comprising providing each of a plurality of plants with a gene editing system, wherein editing by the gene editing system creates an allelic series.

Embodiment 63. The method of any one of embodiments 1-62, further comprising retrieving a progeny of the plant, wherein the progeny has an edited target sequence.

Embodiment 64. An edited plant produced by the method of any one of embodiments 1-63.

Embodiment 65. A progeny of the edited plant of embodiment 64.

1. A method of improving an agronomic trait in a plant or part thereof comprising providing a gene editing system to the plant or part thereof, wherein the gene editing system edits a target site, wherein the target site comprises nucleic acid encoding a translation start site for an upstream open reading frame (uORF) located 5′ to a translation start site for a primary open reading frame (pORF), wherein editing by the gene editing system results in a deletion, substitution, or insertion at the nucleic acid encoding the uORF translation start site.

2. A method of increasing translation of a primary open reading frame (pORF) in a plant or part thereof comprising providing a gene editing system to the plant or part thereof, wherein the gene editing system edits a target site, wherein the target site comprises nucleic acid encoding a translation start site for an upstream open reading frame (uORF) located 5′ to a translation start site for the primary ORF, wherein editing by the gene editing system results in a deletion, substitution, or insertion at the nucleic acid encoding the uORF translation start site.

3. The method of claim 2, wherein editing by the gene editing system results in decreased translation of the uORF.

4. The method of claim 2, wherein protein level of the protein encoded by the primary ORF is increased.

5. A method of improving an agronomic trait in a plant comprising providing a gene editing system to the plant or part thereof, wherein the gene editing system edits a target site, wherein the target site comprises nucleic acid encoding a translation start site for an upstream open reading frame (uORF) located 5′ to a translation start site for a primary open reading frame (pORF), wherein editing by the gene editing system introduces a translation start site for an upstream open reading frame (uORF) in the target site.

6. A method of decreasing translation of a primary open reading frame (ORF) in a plant comprising providing a gene editing system, wherein the gene editing system edits a target site, wherein the target site comprises nucleic acid encoding a translation start site for an upstream open reading frame (uORF) located 5′ to a translation start site for a primary open reading frame (pORF), wherein editing by the gene editing system introduces a translation start site for an upstream open reading frame (uORF) in the target site.

7. The method of claim 6, wherein editing by the gene editing system results in increased translation of the uORF.

8. The method of claim 6, wherein protein level of the protein encoded by the primary ORF is decreased.

9. The method of claim 2, wherein the gene editing system comprises a precise base editing (PBE) system, a PRIME editing system, a CRISPR-Cas9 system, a CRISPR-Cpf1 system, homing endonucleases, a meganuclease, a zinc finger nuclease system, or a transcription activator-like effector nuclease (TALEN) system.

10. The method of claim 2, wherein the gene editing system comprises a Cas endonuclease and a guide RNA for the Cas endonuclease.

11. (canceled)

12. The method of claim 2, wherein the primary ORF encodes a protein involved in drought tolerance, disease resistance, pest resistance, stress tolerance, yield, shape, odor, texture, metabolite production, pigmentation, seed fecundity, endoreduplication, sugar content, pH, improved shelf life or storability, cell differentiation, branching, plant height, growth rates, shoot architecture, root architecture, reproductive organ morphology, abiotic stress tolerance salinity tolerance, heat tolerance, flooding tolerance, resistance or tolerance to biotic stresses, photoperiod sensitivity, time to fruit set, or light reception.

13. (canceled)

14. The method of claim 2, wherein the uORF translation start site is within 500 base pairs of the translation start site for the primary ORF.

15. The method of claim 2, wherein the uORF translation start site comprises a nucleotide sequence selected from the group consisting of AUG, ACG, CUG, UUG, AUA, and AUC.

16. (canceled)

17. The method of claim 2, wherein the uORF translation start site is in-frame with the primary ORF translation start site.

18. The method of claim 2, wherein the uORF translation start site is out-of-frame with the primary ORF translation start site.

19.-20. (canceled)

21. The method of claim 10, comprising providing two or more, three or more, four or more, or five or more guide RNAs to the plant as part of the gene editing system.

22. The method of claim 21, wherein each guide RNA is complementary to a target sequence in a target site, wherein each target site comprises a nucleic acid encoding a uORF translation start site located 5′ to a translation start site for a primary ORF.

23.-25. (canceled)

26. The method of claim 2, wherein two or more, three or more, four or more, or five or more uORFs are removed.

27. The method of claim 2, wherein the gene editing system comprises a Cas endonuclease is selected from the group consisting of Cas9, Cas12a (Cpf1), Cas12e (CasX), Cas12d (CasY), C2c1, C2c2, C2c3, Cas12h, Cas12i, Cas12j, Cas14, and an engineered Cas nuclease.

28.-39. (canceled)

40. The method of claim 2, wherein the plant or part thereof is a crop plant.

41. (canceled)

42. The method of claim 2, wherein the plant or part thereof is selected from the group consisting of maize, rice, sorghum, rye, barley, wheat, pearl millet, foxtail millet, flax, oats, sugarcane, turfgrass, switchgrass, soybean, canola, alfalfa, sunflower, cotton, tobacco, tomato, peanut, potato, cannabis, tomato, a forage crop, an industrial crop, a woody crop, a biomass crop, and Arabidopsis.

43. The method of claim 2, wherein the gene editing system is provided to the plant by transforming the plant with a vector comprising nucleic acid encoding a guide RNA and nucleic acid encoding an endonuclease.

44.-48. (canceled)

49. The method of claim 2, wherein the plant or part thereof comprises a leaf, a shoot, a meristem, a stem, or a root.

50. The method of claim 2, wherein the gene editing system is provided to the plant by application of a composition comprising the gene editing system to the plant or part thereof.

51. The method of claim 50, wherein the gene editing system is provided to the plant by spraying the plant with the composition comprising the gene editing system.

52.-57. (canceled)

58. The method of claim 50, wherein the composition comprising the gene editing system comprises a nuclease inhibitor.

59. The method of claim 58, wherein the nuclease inhibitor comprises an RNase inhibitor.

60-62. (canceled)

63. The method of claim 2, further comprising retrieving a progeny of the plant, wherein the progeny has an edited target sequence.

64. An edited plant produced by the method of claim 2.

65. A progeny of the edited plant of claim 64.
